(19) United States
(12) Patent Application Publication
(10) Pub. No.: US 2014/0363816 A1
(43) Pub. Date: Dec. 11, 2014

(54) METHODS FOR PREDICTION OF CLINICAL RESPONSE TO RADIATION THERAPY IN CANCER PATIENTS

(71) Applicants: THE REGENTS OF THE UNIVERSITY OF COLORADO, a body corporate, Denver, CO (US); THE UNIVERSITY OF VIRGINIA PATENT FOUNDATION, Charlottesville, VA (US)

(72) Inventors: Dan Theodorescu, Englewood, CO (US); Jae K. Lee, Charlottesville, VA (US)

(21) Appl. No.: 14/366,030

(22) PCT Filed: Dec. 21, 2012
(86) PCT No.: PCT/US12/71479

§ 371 Date: Jun. 17, 2014

Related U.S. Application Data
(60) Provisional application No. 61/578,879, filed on Dec. 22, 2011.

(51) Int. Cl. C12Q 1/68

(52) U.S. Cl. CPC C12Q 1/6881; C12Q 2600/106; C12Q 2600/158
USPC 435/6.11

(57) ABSTRACT

Disclosed are biomarkers, methods and assay systems for the identification of cancer patients who are predicted to respond, or not respond to the therapeutic administration of radiation therapy to treat cancer. Thus, the invention provides a diagnostic paradigm to select cancer patients who will benefit from radiation therapy. In particular, the invention provides a novel 41-gene biomarker model associated with clinical outcome following radiotherapy across multiple histological tumor types, including the biomarker Cyclophilin B (PPIB).

### CROSS REFERENCE TO RELATED APPLICATION

The present application claims the benefits of U.S. Provisional Application Serial No. 61/578,879, filed 22 Dec. 2011, which is incorporated herein by this reference in its entirety.

### GOVERNMENT INTEREST

This invention was made with Government support under grant number CA075115 awarded by the National Institutes of Health (NIH). The U.S. Government has certain rights in this invention.

### FIELD OF THE INVENTION

The present invention generally relates to biomarkers, methods and assay kits for the identification of cancer patients predicted to respond to radiation therapy.

### BACKGROUND OF THE INVENTION

Radiation therapy is an important treatment modality for lung, head and neck and bladder cancer, either alone or in combination with chemotherapy. However, the individual response to radiotherapy can be variable and hence any tool that would predict response to this modality would allow enhanced patient stratification among the various treatment options. In addition, once optimally selected, pharmacologic approaches towards radiosensitization promise to further enhance the benefit these patients derive from such treatment. Currently, clinical characteristics of the patient and tumor are primarily used to determine whether treatment with radiotherapy is appropriate while tumor imaging and expression of genes in the tumor tissue have been proposed to possibly enhance this. However, even used together, these are not yet highly predictive of radiation sensitivity of patient tumors before treatment.

Made possible by the development of gene expression microarray or multiplex PCR technologies, mathematical models involving expression measurements of multiple genes have been developed to serve as prognostic indicators of disease aggressiveness or patient survival, and to predict response to specific chemotherapeutic agents or regimens. Associations of tumor gene expression to radiation response have been developed for cell lines and for specific tumors such as cervical cancer, breast cancer, colorectal adenocarcinoma, and cancers of the head and neck. In addition, a radiosensitivity signature as an indicator of concurrent chemoradiation therapeutic response has been tested in small sets of rectal, esophageal and head and neck cancers.

While exciting, the predictive value of these models across different histological tumor types requires validation on larger and more diverse sample sets. In addition, none have identified genes that are both biomarkers and potential targets for radio-sensitization. Thus, there remains a need in the art for sensitive and reliable tools to predict the radiation sensitivity of patient tumors before treatment and thus predict success of radiation therapy and patient outcome.

### DESCRIPTION OF THE DRAWINGS

FIG. 1 shows the selection of the optimal 41-probe model GEM. FIG. 1A shows a plot of the prediction performance of candidate GEMs used to guide selection of optimal GEM. For each candidate GEM the inventors calculated the correlation between the GEM scores and the actual survival fractions for the human fibroblast dataset. The correlation test p-value for each model is plotted vs. the number of genes in the model. The model with 41 genes balances prediction performance and parsimony. FIG. 1B shows normalized survival fraction values and GEM scores on the human fibroblast (HSF) dataset plotted in a bar plot. The Spearman rank-based correlation between normalized GEM score and survival fraction is −0.434, with a one-sided P-Value of 0.0465. Standardization of SF2 and COXEN GEM score involved reciprocal evaluation since higher SF2 indicated radioresistance and higher COXEN GEM score was indicative of radiosensitivity. FIG. 1C shows the evaluation of the predictive ability of the 41-probe GEM in patients with HNSCC. Kaplan-Meier curves for the HNSCC cancer patients stratified into predicted responders and predicted non-responders. Kaplan-Meier curves for patients only treated with radiation (N=72). The left panel shows the overall survival time, while the right panel shows the progression (distant metastasis) free survival time.

FIG. 2 shows the association of gene expression with stage, grade and outcome in lung, bladder and head and neck cancer in box plots showing the association of 7 genes of the 41 gene GEM (Table 3) that were found to be significantly associated with either tumor stage, grade and clinical outcome in lung, bladder and head and neck cancer databases found in Oncomine. P value for the specific comparison and tumor parameter used are indicated in the figure. The datasets which contained these significant associations have been previously published (Beer D G, Kardia S L, Huang C C, Giordano T J, Levin A M, Misek D E, Lin L, Chen G, Gharib T G, Thomas D G, et al. Gene-expression profiles predict survival of patients with lung adenocarcinoma. Nat Med. 2002; 8: 816-824; Bhattacharjee A, Richards W G, Staunton J, Li C, Monti S, Vasa P, Ladd C, Beheshti J, Bueno R, Gillette M, et al. Classification of human lung carcinomas by mRNA expression profiling reveals distinct adenocarcinoma subclasses. Proc Natl Acad Sci USA. 2001; 98: 13790-13795; Bild A H, Yao G, Chang J T, Wang Q, Potti A, Chasse D, Joshi M B, Harpole D, Lancaster J M, Berchuck A, et al. Oncogenic pathway signatures in human cancers as a guide to targeted therapies. Nature. 2006; 439: 353-357; Chung C H, Parker J S, Karaca G, Wu J, Funkhouser W K, Moore D, Butterfoss D, Xiang D, Zanation A, Yin X, et al. Molecular classification of head and neck squamous cell carcinomas using patterns of gene expression. Cancer Cell. 2004; 5: 489-500; Cromer A, Carles A, Millon R, Ganguli G, Channel F, Lemaire F, Young J, Dembele D, Thibault C, Muller D, et al. Identification of genes associated with tumorigenesis and metastatic potential of hypopharyngeal cancer by microarray analysis. Oncogene. 2004; 23: 2484-2498; Dyrskjot L, Thykjaer T, Kruhoffer M, Jensen J L, Marcussen N, Hamilton-Dutoit S, Wolf H, and Orntoft T F. Identifying distinct classes of bladder carcinoma using microarrays. Nat Genet. 2003; 33: 90-96; Ye H, Yu T, Temam S, Ziober B L, Wang J, Schwartz J L, Mao L, Wong D T, and Zhou X. Transcriptomic dissection of tongue squamous cell carcinoma. BMC Genomics. 2008; 9:69).

FIG. 3 shows the effect of Cyclophilin B (PPIB) and Acidic ribosomal phosphoprotein P1 (RPLP1) depletion on in vitro growth and radiosensitivity in human cancer cell lines. FIG. 3A shows an in vitro cell number evaluation using alamarBlue®(Invitrogen, Carlsbad, Calif.) assay following depletion of GL2 (control), PPIB or RPLP1 via siRNA in duplicate samples of UMUC-13d bladder cancer cells, and Western blotting following siRNA depletion of PPIB and RPLP1. Antibodies against PPIB (clone k2e2, Santa Cruz Biotechnology, Inc., Santa Cruz, Calif.) and RPLP1 (polyclonal, Sigma, St. Louis, Mo.) were used. FIG. 3B shows the assessment of apoptosis was assessed by the Annexin V-FITC Apoptosis Detection Kit I (BD Biosciences, Franklin Lakes, N.J.) per the manufacturer's instructions 48 hours following transfection with the siRNA duplexes for PPIB and RPLP1. FIG. 3C shows clonogenic survival in human cancer cell lines following radiation at indicated dose and depletion of GL2 (control), PPIB or RPLP1 via siRNA. * p<0.05 (ANOVA) at 8Gy dose.

FIG. 4 shows the effect of Cyclophilin B (PPIB) depletion and Cyclosporine (CsA) on in vitro growth and radiosensitivity and DNA repair of human bladder cancer cells. FIG. 4A shows an in vitro cell number evaluation using alamarBlue® (Invitrogen, Carlsbad, Calif.) assay following addition of cyclosporin A at indicated doses to duplicate samples of UMUC-13d bladder cancer cells. Arrow indicates dose used in FIGS. 4B-4D. FIG. 4B shows an assessment of apoptosis by the Annexin V-FITC Apoptosis Detection Kit I (BD Biosciences, Franklin Lakes, N.J.) per the manufacturer's instructions 48 hours following transfection with the siRNA duplexes for PPIB and RPLP1 with and without 8 uM cyclosporin A (CsA). FIG. 4C shows clonogenic survival in UMUC-13d bladder cancer cells following radiation at indicated dose and depletion of GL2 (control), PPIB via siRNA with and without 8 uM cyclosporin A (CsA). * p<0.05 (ANOVA) at 8Gy dose. FIG. 4D shows a comet assay following transfection with the siRNA duplex for PPIB as described in Bi. Dose of cyclosporin A (CsA) was 8 uM. Each assay was normalized to cloning efficiency given the apoptosis induced by siRNA depletion. Indicated p values generated using Students T-test. The extent of DNA damage was measured one hour after a 10 Gy exposure.

FIG. 5 shows Cyclophilin B (PPIB) and CDKN2A (p16) expression in HNSCC, and PPIB and p16 immunohistochemistry and their relevance to outcome in HNSCC tumors treated at the University of Virginia (N=72). FIG. 5A shows examples of the IHC scoring of PPIB. Each panel represents a tissue core in a tissue microarray; the numbers 0-3 indicate the expression score given to the tumor in the specimen. Lymphocytes (L) present and infiltrating around the tumor (T) also express this protein at high levels and serve as “internal controls” of staining intensity. FIG. 5B shows Kaplan-Meier curves of overall survival of patients as a function of PPIB immunohistochemistry score. FIG. 5C shows examples of the IHC scoring of p16. Each panel represents a tissue core in a tissue microarray; the characters (+) and (−) indicate the expression score (positive or negative, respectively), given to the tumor in the specimen. FIG. 5D shows Kaplan-Meier curves of overall survival of PPIB positive patients as a function of p16 immunohistochemistry score.

FIG. 6 shows a schematic diagram of gene expression model (GEM) development and validation. FIG. 6A shows the GEM Development, depicting the model selection process. The 955 probe sets not concordantly regulated between the bladder cancer cell line and human bladder cancer patient datasets were first removed from consideration. Then the 300 genes most significantly differentially expressed between radiosensitive and radioresistant bladder cancer cell lines were selected as candidate biomarkers. Models were constructed from these biomarkers, trained on the bladder cancer cell line dataset and used to predict the sensitivity of 16 primary human skin fibroblasts from patients treated with curative intent with radiation. These prediction results were assessed by measuring the correlation between the GEM score and Survival Fraction for the HNSCC lines. The optimal model was found to have 41 probes, and a graph of GEM score vs. survival fraction is shown in FIG. 2A. FIG. 6B shows the GEM Validation process. The optimal model was then used to predict the response to radiation for both the HNSCC and lung cancer patient sets (see Table 1A).

FIG. 7 shows the expression of PPIB and RPLP1 as a function of radiosensitivity: mRNA expression of PPIB and RPLP1 is plotted as a function of 2Gy survival fraction in NCI-60 cell line panel.

### DESCRIPTION OF THE INVENTION

The present inventors have discovered biomarkers that predict clinical outcome following radiation therapy across multiple cancer types. Described herein is a multigene biomarker which is a predictor of clinical outcome following radiation therapy that is applicable across different cancer types. The multigene predictor comprises 41 polynucleotides and polypeptides encoded by them. This predictor provides additional predictive ability even in patients that had other concomitant and potentially confounding treatments such as chemotherapy treatment; yet did not offer any predictive ability in patients that were not treated with radiation. Further described is the biomarker Cyclophilin B (PPIB), a peptidylprolyl isomerase (PPIase) that is strongly related to radiation resistance and is a predictor of clinical outcome in patients with HNSCC. Also described is the biomarker Acidic Ribosomal Phosphoprotein P1 (RPLP1) which is also related to radiation resistance. Depletion of PPIB and RRLP1 was associated with increased radiosensitivity of cancer cells.

The terminology used herein is for describing particular embodiments and is not intended to be limiting. As used herein, the singular forms “a,” “and” and “the” include plural referents unless the content and context clearly dictate otherwise. Thus, for example, a reference to “a marker” may include a combination of two or more such markers. Unless defined otherwise, all scientific and technical terms are to be understood as having the same meaning as commonly used in the art to which they pertain. For the purposes of the present invention, the following terms are defined below.

As used herein, a biological marker (“biomarker” or “marker”) is a characteristic that is objectively measured and evaluated as an indicator of normal biologic processes, pathogenic processes, or pharmacological responses to therapeutic interventions, consistent with NIH Biomarker Definitions Working Group (1998). Markers can also include patterns or ensembles of characteristics indicative of particular biological processes. The biomarker measurement can increase or decrease to indicate a particular biological event or process. In addition, if the biomarker measurement typically changes in the absence of a particular biological process, a constant measurement can indicate occurrence of that process.

The markers of this invention may be used for diagnostic and prognostic purposes, as well as for therapeutic, drug screening and patient stratification purposes (e.g., to group patients into a number of “subsets” for evaluation), as well as other purposes described herein.

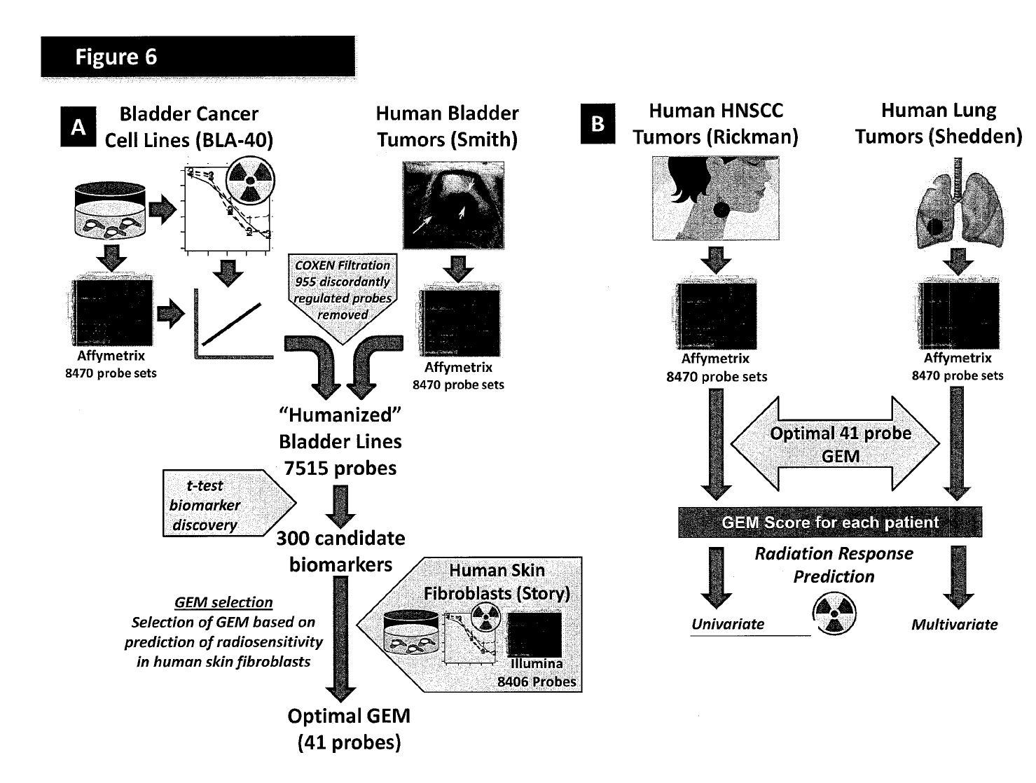

The markers identified for predicting radiosensitivity (or radioresistance) are of significant biologic interest. A schematic of model generation and validation process is depicted in FIG. 6. The methods used are detailed in the Examples section. As explained in Example 1, the COXEN informatic approach was applied to in vitro radiation sensitivity data of transcriptionally profiled human cells and gene expression data from untreated lung, head and neck (HNSCC) and bladder tumors to generate a 41 gene predictive model that is independent of histology and reports on tumor radiosensitivity. This model is also referred herein as Radiation Response Prediction Gene Expression Model (GEM). The predictive ability of this 41-gene model was evaluated in patients with HNSCC and lung adenocarcinoma and was found to stratify clinical outcome following radiotherapy (See Example 2). In contrast, this model was not useful in stratifying similar patients not treated with radiation.

The 41 gene markers of the invention are set forth in Table 3 and are identified by the gene symbol, gene name and the matching probe set. Further included is information related to the function of the gene and the biological process that it is involved in. The polynucleotide sequences of these genes, as well as the sequences of the polypeptides encoded by them are publicly available and known to one having average skill in the art. All information associated with the publicly-available identifiers and accession numbers, including the nucleic acid sequences of the associated genes and the amino acid sequences of the encoded proteins is incorporated herein by reference in its entirety.

Given the name of the protein (also referred to herein as the “full protein”; indicated as “Protein”), other peptide fragments of such measured proteins may be obtained (by whatever means), and such other peptide fragments are included within the scope of the invention. The methods of the present invention may be used to evaluate fragments of the listed molecules as well as molecules that contain an entire listed molecule, or at least a significant portion thereof (e.g., measured unique epitope), and modified versions of the markers. Accordingly, such fragments, larger molecules and modified versions are included within the scope of the invention.

Since the 41 gene GEM was able to stratify clinical outcome following radiotherapy, it was hypothesized that expression of some of the 41 genes contributes to tumor radioresistance and clinical recurrence. Hence the expression the 41 genes was evaluated as a function of in vitro radioresistance in the NCI-60 cancer cell line panel. Cyclophilin B (PPIB) which is a peptidylprolyl isomerase (PPIase), and Acidic Ribosomal Phosphoprotein 1 (RPLP1) were found to have the strongest direct correlation.

The cyclophilins are members of a larger class of PPIase proteins widely expressed throughout the body, known as the immunophilins that are targets for the immunosuppressive agents FK506, cyclosporine A, and rapamycin. PPIB as a secreted protein is also thought to serve as a ligand for the CD147 receptor, thereby regulating the motility of cells expressing this receptor. A study also indicates that PPIB present in the conditioned medium of the MDA-MB-231 breast carcinoma cell line promoted chemotaxis of bone marrow-derived mesenchymal stromal cells.

Expression of Cyclophilin B (PPIB), was found to be strongly related to in vitro radiation response (see Example 4). Depletion of PPIB protein enhanced cell killing after radiation.

Without wishing to be bound by theory, the effect of PPIB was likely by enhancing the apoptotic process, which was phenocopied by exposure of cancer cells to cyclosporine (CsA) which binds PPIB.

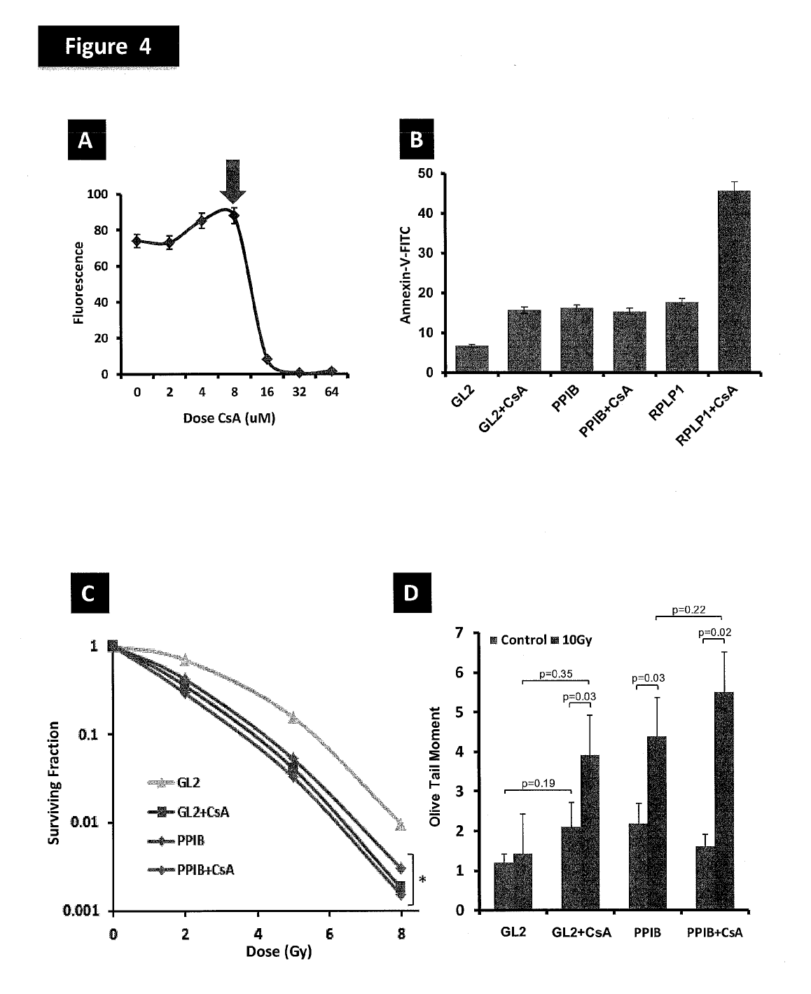

In addition to an enhanced level of apoptosis, there may also be a role for DNA repair via PPIB knockdown. CsA binds and inhibits PPIB thus interfering with DNA repair by decreasing calcineurin-mediated expression of DNA polymerase β. Using a dominant negative form of polymerase β after ionizing radiation, cell cycle position-dependent radiosensitization, higher numbers of chromatid-type aberrations that result in replication-dependent secondary DNA double-strand breaks, and a higher number of chromosomal deletions were all seen, and the chromosomal deletions were described as the mechanism of enhanced cell killing. Also supporting the notion of reduced DNA repair capacity are the results from the comet assay that measures both single- and double-strand breaks. The induction of single-strand breaks occurs at a frequency of almost two orders of magnitude over double-strand breaks and is ordinarily rapidly repaired. Within 1 hour, more than 90% of the total strand breaks would be repaired as was seen in FIG. 4C. Although it is residual DNA lesions that drive cell death, given the extent of apoptosis seen at 24 hours after irradiation, it is conceivable that unrepaired DNA lesions, stalled replication forks, and others, may initiate the apoptotic process in cells compromised by exposure to CsA or reduced PPIB.

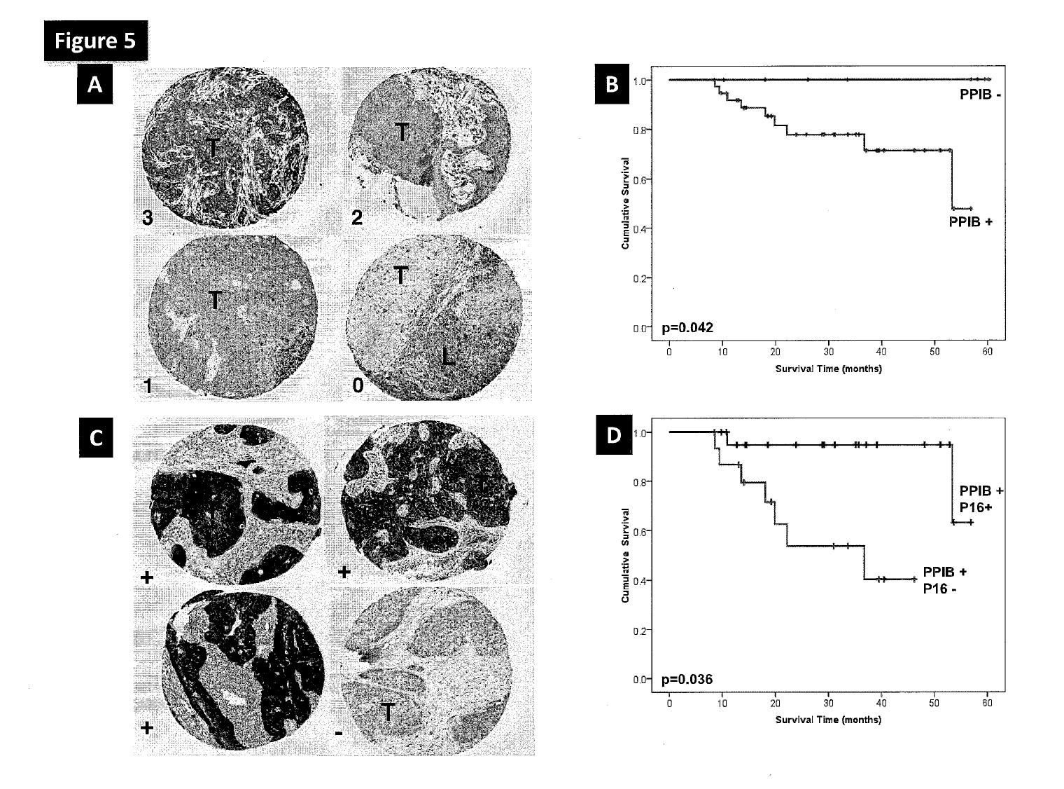

Furthermore, expression of Cyclophilin B (PPIB), was found to be a predictor of clinical outcome in patients with HNSCC (see Example 5). Given that PPIB RNA expression was strongly correlated to radioresistance, the role of PPIB protein expression was evaluated as a predictor of clinical outcome following radiation treatment of patients with HNSCC at the University of Virginia. PPIB protein levels were found to predict clinical outcome in these patients (FIG. 5A, B).

Expression of CDKN2A (p16), a cyclin-dependent kinase inhibitor and surrogate marker of HPV infection was recently found to predict radiation response in patients with HNCCC. Because this gene was part of the signaling network associated with the 41-gene GEM (Table 3), the inventors sought to determine whether its level of protein expression provided additional predictive ability when combined with that of PPIB. IHC evaluation revealed that p16 levels provided significant stratification of patients with high PPIB IHC (FIGS. 5, C and D) levels supporting relevance of the 39-gene network in radiosensitivity of human cancer.

Thus the finding reported herein identify Cyclophilin B or PPIB as both a novel biomarker of outcome following radiation therapy and a potential therapeutic target for improving the effects of radiation therapy.

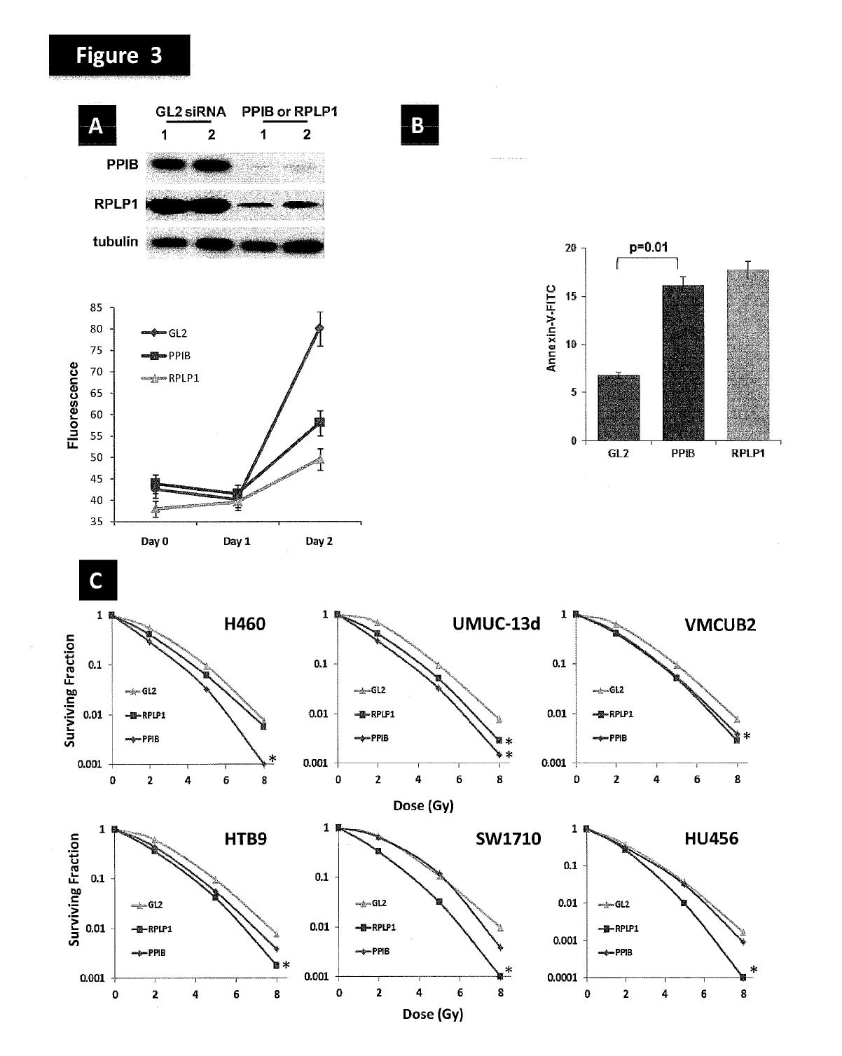

Additionally, reported herein is the protein RPLP1, whose expression is associated with radiosensitivity. Reduced levels of RPLP1 were associated with reduction in cell number following depletion (FIG. 3A), likely due to enhanced apoptosis (FIG. 3B). When 6 human cancer cell lines were transiently depleted of either RPLP1 and irradiated it was noted that cells with reduced levels of RPLP1 had reduced clonogenicity (FIG. 3C).

The present invention includes all compositions and methods relying on correlations between the reported biomarkers and the radiosensitivity (or radioresistance) of the cancer cells. Such methods include methods for determining whether a cancer patient is predicted to respond to administration of radiation therapy, as well as methods for assessing the efficacy of a radiation therapy.

Further included are methods for improving the efficacy of a radiation therapy by administering to a subject a therapeutically effective amount of an agent that inhibits the activity or expression of a biomarker, such as Cyclophilin B (PPIB). In this context, the term “effective” is to be understood broadly to include reducing or alleviating the signs or symptoms of cancer, improving the clinical course of the disease, or reducing any other objective or subjective indicia of the disease. Different drugs, doses and delivery routes can be evaluated by performing the method using different drug administration conditions. The markers may also be used as pharmaceutical compositions or in kits. The markers may also be used to screen candidate compounds that modulate their expression.

It is expected that the biomarkers described herein will be measured in combination with other signs, symptoms and clinical tests of cancer, such as skin examination, dermoscopy, lymph node examination, chest x-ray, CT scan of the chest, head, abdomen, or pelvis, magnetic resonance imaging (MRI), and/or serum lactate dehydrogenase blood tests. Measurement of the biomarkers of the invention along with any other marker known in the art, including those not specifically listed herein, falls within the scope of the present invention.

Marker measurements may be of the absolute values (e.g., the molar concentration of a molecule in a biological sample) or relative values (e.g., the relative concentration of two molecules in a biological sample). The quotient or product of two or more measurements also may be used as a marker. For example, some physicians use the total blood cholesterol as a marker of the risk of developing coronary artery disease, while others use the ratio of total cholesterol to HDL cholesterol.

The term “including” is used herein to mean, and is used interchangeably with, the phrase “including but not limited to.” The term “or” is used herein to mean, and is used interchangeably with, the term “and/or,” unless context clearly indicates otherwise

As used herein, the phrase “gene expression” or “protein expression” includes any information pertaining to the amount of gene transcript or protein present in a sample, as well as information about the rate at which genes or proteins are produced or are accumulating or being degraded (e.g., reporter gene data, data from nuclear runoff experiments, pulse-chase data etc.). Certain kinds of data might be viewed as relating to both gene and protein expression. For example, protein levels in a cell are reflective of the level of protein as well as the level of transcription, and such data is intended to be included by the phrase “gene or protein expression information.” Such information may be given in the form of amounts per cell, amounts relative to a control gene or protein, in unitless measures, etc.; the term “information” is not to be limited to any particular means of representation and is intended to mean any representation that provides relevant information. The term “expression levels” refers to a quantity reflected in or derivable from the gene or protein expression data, whether the data is directed to gene transcript accumulation or protein accumulation or protein synthesis rates, etc.

The practice of the invention employs, unless otherwise indicated, conventional methods of analytical biochemistry, microbiology, molecular biology and recombinant DNA techniques generally known within the skill of the art. Such techniques are explained fully in the literature. (See, e.g., Sambrook et al. Molecular Cloning: A Laboratory Manual. 3rd, ed., Cold Spring Harbor Laboratory, Cold Spring Harbor Laboratory Press, Cold Spring Harbor, N.Y., 2000; DNA Cloning: A Practical Approach, Vol. I & II (Glover, ed.); Oligonucleotide Synthesis (Gait, ed., Current Edition); Nucleic Acid Hybridization (Hames & Higgins, eds., Current Edition); Transcription and Translation (Hames & Higgins, eds., Current Edition); CRC Handbook of Parvoviruses, Vol. I & II (Tijessen, ed.); Fundamental Virology, 2nd Edition, Vol. I & II (Fields and Knipe, eds.)).

As used herein, a component (e.g., a marker) is referred to as “differentially expressed” in one sample as compared to another sample when the method used for detecting the component provides a different level or activity when applied to the two samples. A component is referred to as “increased” or “upregulated” in the first sample if the method for detecting the component indicates that the level or activity of the component is higher in the first sample than in the second sample (or if the component is detectable in the first sample but not in the second sample). Conversely, a component is referred to as “decreased” or “downregulated” in the first sample if the method for detecting the component indicates that the level or activity of the component is lower in the first sample than in the second sample (or if the component is detectable in the second sample but not in the first sample). In particular, marker is referred to as “increased” (“upregulated”) or “decreased” (“downregulated”) in a sample (or set of samples) obtained from a cancer subject (or a subject who is suspected of having cancer, or is at risk of developing cancer) if the level or activity of the marker is higher or lower, respectively, compared to the level of the marker in a sample (or set of samples) obtained from a non-cancer subject, or a reference value or range.

As used herein, a compound is referred to as “isolated” when it has been separated from at least one component with which it is naturally associated. For example, a metabolite can be considered isolated if it is separated from contaminants including polypeptides, polynucleotides and other metabolites. Isolated molecules can be either prepared synthetically or purified from their natural environment. Standard quantification methodologies known in the art can be employed to obtain and isolate the molecules of the invention.

Homologs and alleles of the polypeptide markers of the invention can be identified by conventional techniques. As used herein, a homolog to a polypeptide is a polypeptide from a human or other animal that has a high degree of structural similarity to the identified polypeptides. Identification of human and other organism homologs of polypeptide markers identified herein will be familiar to those of skill in the art. In general, nucleic acid hybridization is a suitable method for identification of homologous sequences of another species (e.g., human, cow, sheep), which correspond to a known sequence. Standard nucleic acid hybridization procedures can be used to identify related nucleic acid sequences of selected percent identity. For example, one can construct a library of cDNAs reverse transcribed from the mRNA of a selected tissue (e.g., colon) and use the nucleic acids that encode polypeptides identified herein to screen the library for related nucleotide sequences. The screening preferably is performed using high-stringency conditions (described elsewhere herein) to identify those sequences that are closely related by sequence identity. Nucleic acids so identified can be translated into polypeptides and the polypeptides can be tested for activity.

Some variation is inherent in the measurements of the physical and chemical characteristics of the markers. The magnitude of the variation depends to some extent on the reproducibility of the separation means and the specificity and sensitivity of the detection means used to make the measurement. Preferably, the method and technique used to measure the markers is sensitive and reproducible.

Polypeptides encoded by the gene markers identified in Table 3 reflect a single polypeptide appearing in a database. In general, the polypeptide is the largest polypeptide found in the database. But such a selection is not meant to limit the polypeptide to those corresponding to those single polypeptides. Accordingly, in another embodiment, the invention provides a polypeptide that is a fragment, precursor, successor or modified version of a marker described in Table 3. In another embodiment, the invention includes a molecule that comprises a foregoing fragment, precursor, successor or modified polypeptide.

As used herein, a “fragment” of a polypeptide refers to a single amino acid or a plurality of amino acid residues comprising an amino acid sequence that has at least 5 contiguous amino acid residues, at least 10 contiguous amino acid residues, at least 20 contiguous amino acid residues or at least 30 contiguous amino acid residues of a sequence of the polypeptide. As used herein, a “fragment” of polynucleotide refers to a single nucleic acid or to a polymer of nucleic acid residues comprising a nucleic acid sequence that has at least 15 contiguous nucleic acid residues, at least 30 contiguous nucleic acid residues, at least 60 contiguous nucleic acid residues, or at least 90% of a sequence of the polynucleotide. In some embodiment, the fragment is an antigenic fragment, and the size of the fragment will depend upon factors such as whether the epitope recognized by an antibody is a linear epitope or a conformational epitope. Thus, some antigenic fragments will consist of longer segments while others will consist of shorter segments, (e.g. 5, 6, 7, 8, 9, 10, 11 or 12 or more amino acids long, including each integer up to the full length of the polypeptide). Those skilled in the art are well versed in methods for selecting antigenic fragments of proteins.

In some embodiments, a polypeptide marker is a member of a biological pathway. As used herein, the term “precursor” or “successor” refers to molecules that precede or follow the polypeptide marker or polynucleotide marker in the biological pathway. Thus, once a polypeptide marker or polynucleotide marker is identified as a member of one or more biological pathways, the present invention can include additional precursor or successor members of the biological pathway. Such identification of biological pathways and their members is within the skill of one in the art.

Additionally, the present invention includes polypeptides that have substantially similar sequence identity to the polypeptides of the present invention. As used herein, two polypeptides have “substantial sequence identity” when there is at least about 70% sequence identity, at least about 80% sequence identity, at least about 90% sequence identity, at least about 95% sequence identity, at least about 99% sequence identity, and preferably 100% sequence identity between their amino acid sequences, or when polynucleotides encoding the polypeptides are capable of forming a stable duplex with each other under stringent hybridization conditions. For example, conservative amino acid substitutions may be made in polypeptides to provide functionally equivalent variants of the foregoing polypeptides, i.e., the variants retain the functional capabilities of the polypeptides. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods.

As used herein, the term “gene” or “polynucleotide” refers to a single nucleotide or a polymer of nucleic acid residues of any length. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. For example a polynucleotide includes a gene, a gene fragment, cDNA, isolated DNA, mRNA, tRNA, rRNA, isolated RNA of any sequence, recombinant polynucleotides, primers, probes, plasmids, and vectors. Included within the definition are nucleic acid polymers that have been modified, whether naturally or by intervention.

In another embodiment, the invention provides polynucleotides that have substantial sequence similarity to a polynucleotide that is described in Table A. Two polynucleotides have “substantial sequence identity” when there is at least about 70% sequence identity, at least about 80% sequence identity, at least about 90% sequence identity, at least about 95% sequence identity or at least 99% sequence identity between their amino acid sequences or when the polynucleotides are capable of forming a stable duplex with each other under stringent hybridization conditions. Such conditions are well known in the art. As described above with respect to polypeptides, the invention includes polynucleotides that are allelic variants, the result of SNPs, or that in alternative codons to those present in the native materials as inherent in the degeneracy of the genetic code.

In some embodiments, the methods comprise detecting in a sample of tumor cells from a patient, a level of gene expression of one or more biomarkers, wherein the expression levels of the markers are indicative of whether the patient will respond to the administration of radiation therapy.

As used herein, the term “sample” includes a sample from any body fluid or tissue (e.g., serum, plasma, blood, cerebrospinal fluid, urine, saliva, cancer tissue).

As used herein, the terms “patient,” “subject” and “a subject or patient who has cancer” and “cancer patient or subject” are intended to refer to subjects who have been diagnosed with cancer. The terms “normal,” “normal control” and “a subject who does not have cancer” are intended to refer to a subject who has not been diagnosed with cancer, or who is cancer-free as a result of surgery to remove the diseased tissue. A non-cancer subject may be healthy and have no other disease, or they may have a disease other than cancer. A “subject” is any organism of interest, generally a mammalian subject, such as a mouse, and preferably a human subject.

The markers of the invention are useful for predicting outcome of radiation in multiple cancer types, including without limitation, bladder cancer, lung cancer, head and neck cancer, glioma, gliosarcoma, anaplastic astrocytoma, medulloblastoma, lung cancer, small cell lung carcinoma, cervical carcinoma, colon cancer, rectal cancer, chordoma, throat cancer, Kaposi's sarcoma, lymphangiosarcoma, lymphangioendotheliosarcoma, colorectal cancer, endometrium cancer, ovarian cancer, breast cancer, pancreatic cancer, prostate cancer, renal cell carcinoma, hepatic carcinoma, bile duct carcinoma, choriocarcinoma, seminoma, testicular tumor, Wilms' tumor, Ewing's tumor, bladder carcinoma, angiosarcoma, endotheliosarcoma, adenocarcinoma, sweat gland carcinoma, sebaceous gland sarcoma, papillary sarcoma, papillary adenosarcoma, cystadenosarcoma, bronchogenic carcinoma, medullary carcinoma, mastocytoma, mesotheliorma, synovioma, melanoma, leiomyosarcoma, rhabdomyosarcoma, neuroblastoma, retinoblastoma, oligodentroglioma, acoustic neuroma, hemangioblastoma, meningioma, pinealoma, ependymoma, craniopharyngioma, epithelial carcinoma, embryonal carcinoma, squamous cell carcinoma, base cell carcinoma, fibrosarcoma, myxoma, myxosarcoma, liposarcorna, chondrosarcoma, osteogenic sarcoma, and leukemia. In some embodiments the cancer may be bladder cancer, lung cancer or head and neck cancer.

The markers may be detected by any method known to those of skill in the art, including without limitation LC-MS, GC-MS, immunoassays, hybridization and enzyme assays. The detection may be quantitative or qualitative. A wide variety of conventional techniques are available, including mass spectrometry, chromatographic separations, 2-D gel separations, binding assays (e.g., immunoassays), competitive inhibition assays, and so on. Any effective method in the art for measuring the presence/absence, level or activity of a marker is included in the invention. It is within the ability of one of ordinary skill in the art to determine which method would be most appropriate for measuring a specific marker. Thus, for example, an ELISA assay may be best suited for use in a physician's office while a measurement requiring more sophisticated instrumentation may be best suited for use in a clinical laboratory. Regardless of the method selected, it is important that the measurements be reproducible.

The markers of the invention can be detected and measured by mass spectrometry, which allows direct measurements of analytes with high sensitivity and reproducibility. A number of mass spectrometric methods are available. As will be appreciated by one of skill in the art, many separation technologies may be used in connection with mass spectrometry. For example, a wide selection of separation columns is commercially available. In addition, separations may be performed using custom chromatographic surfaces (e.g., a bead on which a marker specific reagent has been immobilized). Molecules retained on the media subsequently may be eluted for analysis by mass spectrometry.

In one embodiment, the expression of the marker genes is detected by detecting the presence of transcripts of the gene in cells in a biological sample. The expression of the marker genes may be detected by detecting hybridization of at least a portion of the gene or a transcript thereof, to a nucleic acid molecule comprising a portion of the gene and a transcript thereof in a nucleic acid array. The expression of the marker genes may also be detected by obtaining RNA from the cancer tissue sample; generating cDNA from the RNA; amplifying the cDNA with probes or primers for marker genes; and obtaining from the amplified cDNA the expression levels of the genes or gene expression products in the sample

In another aspect the expression of the marker genes is detected by detecting the production of polypeptides encoded by the marker genes. The polypeptides may be detected by using a reagent that specifically binds to the polypeptide or a fragment thereof.

The present invention also encompasses reagents or molecules which specifically bind the markers. As used herein, the term “specifically binding,” refers to the interaction between binding pairs (e.g., an antibody and an antigen or aptamer and its target). In some embodiments, the interaction has an affinity constant of at most 10⁻⁶ moles/liter, at most 10⁻⁷ moles/liter, or at most 10⁻⁸ moles/liter. In other embodiments, the phrase “specifically binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

The binding molecules include antibodies, aptamers and antibody derivatives or fragments. As used herein, the term “antibody” refers to an immunoglobulin molecule capable of binding an epitope present on an antigen. The term is intended to encompass not only intact immunoglobulin molecules such as monoclonal and polyclonal antibodies, but also bi-specific antibodies, humanized antibodies, chimeric antibodies, anti-idiopathic (anti-ID) antibodies, single-chain antibodies, Fab fragments, F(ab′) fragments, fusion proteins and any modifications of the foregoing that comprise an antigen recognition site of the required specificity.

As used herein, an aptamer is a non-naturally occurring nucleic acid molecule or peptide having a desirable action on a target, including, but not limited to, binding of the target, catalytically changing the target, reacting with the target in a way which modifies/alters the target or the functional activity of the target, covalently attaching to the target as in a suicide inhibitor, facilitating the reaction between the target and another molecule.

In one embodiment, the antibodies, antibody derivatives or fragments, or aptamers specifically bind to a component that is a fragment, modification, precursor or successor of a marker.

Certain antibodies that specifically bind markers of the invention are already known and/or available for purchase from commercial sources. The antibodies of the invention may also be prepared by any suitable means known in the art. For example, antibodies may be prepared by immunizing an animal host with the marker or an immunogenic fragment thereof (conjugated to a carrier, if necessary). Adjuvants (e.g., Freund's adjuvant) optionally may be used to increase the immunological response. Sera containing polyclonal antibodies with high affinity for the antigenic determinant can then be isolated from the immunized animal and purified. Alternatively, antibody-producing tissue from the immunized host can be harvested and a cellular homogenate prepared from the organ can be fused to cultured cancer cells. Hybrid cells which produce monoclonal antibodies specific for a marker can be selected. Alternatively, the antibodies of the invention can be produced by chemical synthesis or by recombinant expression. For example, a polynucleotide that encodes the antibody can be used to construct an expression vector for the production of the antibody. The antibodies of the present invention can also be generated using various phage display methods known in the art.

Antibodies or aptamers that specifically bind the markers can be used, for example, in methods for detecting levels of marker using methods and techniques well-known in the art. In some embodiments, for example, the antibodies are conjugated to a detection molecule or moiety (e.g., a dye, and enzyme) and can be used in ELISA or sandwich assays to detect markers of the invention.

In another embodiment, antibodies or aptamers against a marker can be used to assay a tissue sample for the marker. The antibodies or aptamers can specifically bind to the marker, if any, present in the tissue sections and allow the localization of the marker in the tissue. Similarly, antibodies or aptamers labeled with a radioisotope may be used for in vivo imaging or treatment applications.

Another aspect of the invention provides compositions comprising the marker, a binding molecule that is specific for the marker (e.g., an antibody or an aptamer), an inhibitor of the marker, or other molecule that can increase or decrease the level or activity of the marker. Such compositions may be pharmaceutical compositions formulated for use as a therapeutic. Alternatively, the invention provides a composition that comprises a component that is a fragment, modification, precursor, or successor of a marker or a molecule that comprises a foregoing component.

In another embodiment, the invention provides a composition that comprises an antibody or aptamer that specifically binds to a marker polypeptide or a molecule that comprises a foregoing antibody or aptamer.

In some embodiments, the level of the markers may be determined using a standard immunoassay, such as sandwiched ELISA using matched antibody pairs and chemiluminescent detection.

Commercially available or custom monoclonal or polyclonal antibodies are typically used. However, the assay can be adapted for use with other reagents that specifically bind to the marker. Standard protocols and data analysis are used to determine the marker concentrations from the assay data. The binding molecules may be identified and produced by any method accepted in the art. Methods for identifying and producing antibodies and antibody fragments specific for a polypeptide are well known. Examples of other methods used to identify the binding molecules include binding assays with random peptide libraries (e.g., phage display) and design methods based on an analysis of the structure of a biomarker.

The markers of the invention also may be detected or measured using a number of chemical derivatization or reaction techniques known in the art. Reagents for use in such techniques are known in the art, and are commercially available for certain classes of target molecules.

Finally, the chromatographic separation techniques described above also may be coupled to an analytical technique other than mass spectrometry such as fluorescence detection of tagged molecules, NMR, capillary UV, evaporative light scattering or electrochemical detection.

Typical methodologies for protein detection include protein extraction from a cell or tissue sample, followed by hybridization of a labeled probe (e.g., an antibody) specific for the target protein to the protein sample, and detection of the probe. The label group can be a radioisotope, a fluorescent compound, an enzyme, or an enzyme co-factor. Detection of specific protein and polynucleotides may also be assessed by gel electrophoresis, column chromatography, direct sequencing, or quantitative PCR (in the case of polynucleotides) among many other techniques well known to those skilled in the art.

Detection of the presence or number of copies of all or a part of a marker gene of the invention may be performed using any method known in the art. Typically, it is convenient to assess the presence and/or quantity of a DNA or cDNA by Southern analysis, in which total DNA from a cell or tissue sample is extracted, is hybridized with a labeled probe (e.g., a complementary DNA molecule), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as is known by one skilled in the art.

Polynucleotide similarity can be evaluated by hybridization between single stranded nucleic acids with complementary or partially complementary sequences. Such experiments are well known in the art. High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10 C less than for DNA:RNA hybrids. In particular embodiments, stringent hybridization conditions for DNA:DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42 C, followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC). Other hybridization conditions, and for example, those most useful with nucleic acid arrays, will be known to those of skill in the art.

In some embodiments, the level of the markers is compared to a standard level or a reference level. Typically, the standard biomarker level or reference range is obtained by measuring the same marker or markers in a set of normal controls. Measurement of the standard biomarker level or reference range need not be made contemporaneously; it may be a historical measurement. Preferably the normal control is matched to the patient with respect to some attribute(s) (e.g., age). Depending upon the difference between the measured and standard level or reference range, the patient can be diagnosed as predicted to respond to the radiation therapy or as not predicted to respond to the radiation therapy.

The plurality of biomarkers includes at least two or more biomarkers (e.g., at least 2, 3, 4, 5, 6, and so on, in whole integer increments, up to all of the possible biomarkers) identified by the present invention, and includes any combination of such biomarkers. Such markers are selected from any of the polynucleotides listed in the table 3 provided herein, and polypeptides encoded by any of them. In some embodiments, the plurality of markers includes the marker PPIB or the marker RPLP1 or both and at least one other marker listed in Table 3. In some embodiments, the plurality of markers includes the marker PPIB and CDKN2A gene and at least one other marker from Table 3. In one embodiment, the plurality of markers used in the present invention includes all of the markers in the gene signature that has been demonstrated to be predictive of response to the radiation therapy in a cancer patient.

In some embodiments, only the marker PPIB, or RPLP1 are included. In some embodiments, the marker PPIB and the gene CDKN2A are included.

In various preferred embodiments, the marker or plurality of the markers includes i) a single marker gene having at least 95% sequence identity with PPIB gene or RPLP1 gene; or homologs or variants thereof; ii) a plurality of marker genes comprising a marker gene having at least 95% sequence identity to PPIB gene and another marker gene having at least 95% sequence identity to CDKN2A gene, or homologs or variants thereof; iii) a plurality of marker genes comprising a marker gene having at least 95% sequence identity with PPIB gene or RPLP1 gene or both, and at least one marker gene having at least 95% sequence identity with a sequence selected from table 3, or homologs or variants thereof; iv) a plurality of marker genes comprising a marker gene having at least 95% sequence identity with PPIB, a marker gene having at least 95% sequence identity with CDKN2A and at least one marker gene having at least 95% sequence identity with a sequence selected from Table 3, or homologs or variants thereof; and v) a plurality of marker genes having at least 95% sequence identity with a sequence selected from table 3, or homologs or variants thereof. In some embodiments, the marker gene or the plurality of the marker genes includes polynucleotides that are fully complimentary to the at least a portion of the genes from (i)-(v) of the previous statement.

In an alternative embodiment of the invention, a method is provided for assessing the efficacy or effectiveness of a radiation treatment being administered to a cancer patient. The specific techniques used in implementing this embodiment are similar to those used in the embodiments described above. The method is performed by obtaining a first sample, such as serum or tissue, from the subject at a certain time (t₀); measuring the level of at least one of the biomarkers in the biological sample; and comparing the measured level with the level measured with respect to a sample obtained from the subject at a later time (t₁). Depending upon the difference between the measured levels, it can be seen whether the marker level has increased, decreased, or remained constant over the interval (t₁−t₀). Subsequent sample acquisitions and measurements can be performed as many times as desired over a range of times t₂ to tₙ.

If the biomarker is PPIB or RPLP1 then a decrease in the marker level would indicate that the radiation therapy is successful in killing cancer cells. On the other hand, an increase in the marker level would indicate that the radiation therapy is not successful in killing cancer cells and the amount and/or duration of radiation exposure may be increased.

In another aspect, the invention provides an assay system or kit for predicting patient response or outcome to a radiation therapy for cancer, comprising a means for detecting the expression of at least one marker gene or plurality of marker genes in a sample from a subject.

The kits of the invention may comprise one or more of the following: an antibody, wherein the antibody specifically binds with a marker of the present invention, a labeled binding partner to the antibody, a solid phase upon which is immobilized the antibody or its binding partner, instructions on how to use the kit, and a label or insert indicating regulatory approval for diagnostic or therapeutic use.

The kit may also include microarrays comprising a marker, or molecules, such as antibodies, which specifically bind to the marker. In this aspect of the invention, standard techniques of microarray technology are utilized to assess expression of the marker polypeptides and/or identify biological constituents that bind such polypeptides. Protein microarray technology is well known to those of ordinary skill in the art and is based on, but not limited to, obtaining an array of identified peptides or proteins on a fixed substrate, binding target molecules or biological constituents to the peptides, and evaluating such binding.

The assay system preferably also includes one or more controls. The controls may include: (i) information containing a predetermined control level of the marker gene that has been correlated with response to the administration of radiation therapy; and (ii) information containing a predetermined control level of the marker gene that has been correlated with lack of response to the administration of radiation therapy.

In another embodiment, a means for detecting the expression level of a marker or markers can generally be any type of reagent that can include, but are not limited to, antibodies and antigen binding fragments thereof, peptides, binding partners, aptamers, enzymes, and small molecules. Additional reagents useful for performing an assay using such means for detection can also be included, such as reagents for performing immunohistochemistry or another binding assay.

The means for detecting of the assay system of the present invention can be conjugated to a detectable tag or detectable label. Such a tag can be any suitable tag which allows for detection of the reagents used to detect the marker and includes, but is not limited to, any composition or label detectable by spectroscopic, photochemical, electrical, optical or chemical means. Useful labels in the present invention include: biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

In addition, the means for detecting of the assay system of the present invention can be immobilized on a substrate. Such a substrate can include any suitable substrate for immobilization of a detection reagent such as would be used in any of the previously described methods of detection. Briefly, a substrate suitable for immobilization of a means for detecting includes any solid support, such as any solid organic, biopolymer or inorganic support that can form a bond with the means for detecting without significantly affecting the activity and/or ability of the detection means to detect the desired target molecule. Exemplary organic solid supports include polymers such as polystyrene, nylon, phenol-formaldehyde resins, and acrylic copolymers (e.g., polyacrylamide). The kit can also include suitable reagents for the detection of the reagent and/or for the labeling of positive or negative controls, wash solutions, dilution buffers and the like. The assay system can also include a set of written instructions for using the system and interpreting the results.

The assay system can also include a means for detecting a control marker that is characteristic of the cell type being sampled and can generally be any type of reagent that can be used in a method of detecting the presence of a known marker (at the nucleic acid or protein level) in a sample, such as by a method for detecting the presence of a biomarker described previously herein. Specifically, the means is characterized in that it identifies a specific marker of the cell type being analyzed that positively identifies the cell type. Such a means increases the accuracy and specificity of the assay of the present invention. Such a means for detecting a control marker include, but are not limited to: a probe that hybridizes under stringent hybridization conditions to a nucleic acid molecule encoding a protein marker; PCR primers which amplify such a nucleic acid molecule; an aptamer that specifically binds to a conformationally-distinct site on the target molecule; and/or an antibody, antigen binding fragment thereof, or antigen binding peptide that selectively binds to the control marker in the sample. Nucleic acid and amino acid sequences for many cell markers are known in the art and can be used to produce such reagents for detection.

In another aspect, the invention provides methods for improving the response of a cancer patient to radiation therapy. The methods comprise administering a therapeutically effective amount of at least one agent that inhibits the activity of expression of protein cyclophilin B (PPIB).

In some embodiments, the agent may be administered prior to the administration of the radiation therapy i.e. prior to administering or commencing the radiation therapy. In some embodiments, the agent may be administered simultaneously with or at the same time as the administration of the radiotherapy.

As used herein, the term “agent” means a chemical or biological molecule such as a simple or complex organic molecule, a peptide, a polypeptide or protein, or a nucleic acid molecule that is able to inhibit the expression or activity of the PPIB protein. Such molecules may be purchased commercially or synthesized using methods known in the art.

Suitable organic molecules to be used as agents may include drugs, synthetic or naturally occurring, that are capable of inhibiting the activity of the PPIB protein.

In some embodiments, the agent may be a polypeptide or protein. In one aspect, the protein is an antibody specifically reactive with a PPIB protein or polypeptide that is effective for decreasing a biological activity of the PPIB protein or polypeptide. For example, by using immunogens derived from a PPIB protein or polypeptide, e.g., based on the cDNA sequences, anti-protein/anti-peptide antisera or monoclonal antibodies can be made by standard protocols (See, for example, Antibodies: A Laboratory Manual ed. by Harlow and Lane (Cold Spring Harbor Press: 1988)). Such methods are well known in the art and have also been discussed before in this application.

A mammal, such as a mouse, a hamster or rabbit can be immunized with an immunogenic form of the PPIB (e.g., PPIB protein or polypeptide or an antigenic fragment which is capable of eliciting an antibody response, or a fusion protein). Techniques for conferring immunogenicity on a protein or peptide include conjugation to carriers or other techniques well known in the art. An immunogenic portion of a PPIB protein or polypeptide can be administered in the presence of adjuvant. The progress of immunization can be monitored by detection of antibody titers in plasma or serum. Standard ELISA or other immunoassays can be used with the immunogen as antigen to assess the levels of antibodies. In a preferred embodiment, the subject antibodies are immunospecific for antigenic determinants of a PPIB protein or polypeptide of a mammal. Following immunization of an animal with an antigenic preparation of PPIB protein or polypeptide, anti-PPIB antisera can be obtained and, if desired, polyclonal anti-PPIB antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Again, such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a mammalian PPIB protein or polypeptide and monoclonal antibodies isolated from a culture comprising such hybridoma cells.

In certain preferred embodiments, an antibody of the invention is a monoclonal antibody, and in certain embodiments the invention makes available methods for generating novel antibodies. For example, a method for generating a monoclonal antibody that binds specifically to a PPIB protein or polypeptide may comprise administering to a mouse an amount of an immunogenic composition comprising the PPIB protein or polypeptide effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monoclonal antibody that binds specifically to the PPIB protein or polypeptide. Once obtained, a hybridoma can be propagated in a cell culture, optionally in culture conditions where the hybridoma-derived cells produce the monoclonal antibody that binds specifically to the PPIB protein or polypeptide. The monoclonal antibody may be purified from the cell culture.

One characteristic that influences the specificity of an antibody:antigen interaction is the affinity of the antibody for the antigen. Although the desired specificity may be reached with a range of different affinities, generally preferred antibodies will have an affinity (a dissociation constant) of about 10⁻⁶, 10⁻⁷, 10⁻⁸, 10⁻⁹ or less.

In addition, the techniques used to screen antibodies in order to identify a desirable antibody may influence the properties of the antibody obtained. For example, an antibody to be used for certain therapeutic purposes will preferably be able to target a particular cell type. Accordingly, to obtain antibodies of this type, it may be desirable to screen for antibodies that bind to cells that express the antigen of interest (e.g., by fluorescence activated cell sorting). Likewise, if an antibody is to be used for binding an antigen in solution, it may be desirable to test solution binding. A variety of different techniques are available for testing antibody:antigen interactions to identify particularly desirable antibodies. Such techniques include ELISAs, surface plasmon resonance binding assays (e.g., the Biacore binding assay, Bia-core AB, Uppsala, Sweden), sandwich assays (e.g., the paramagnetic bead system of IGEN International, Inc., Gaithersburg, Md.), western blots, immunoprecipitation assays and immunohistochemistry.

In some embodiments, the agent may be a nucleic acid molecule. In certain aspects, the nucleic acid molecule may be RNAi, ribozyme, antisense, DNA enzyme or other nucleic acid-related compositions for manipulating (typically decreasing) PPIB expression or activity.

Some embodiments of the invention make use of materials and methods for effecting knockdown of PPIB gene by means of RNA interference (RNAi). RNAi is a process of sequence-specific post-transcriptional gene repression which can occur in eukaryotic cells. In general, this process involves degradation of an mRNA of a particular sequence induced by double-stranded RNA (dsRNA) that is homologous to that sequence. Any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Accordingly, RNAi may be effected by introduction or expression of relatively short homologous dsRNAs.

The double stranded oligonucleotides used to effect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides of the invention may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al. (2001) Nature 411: 494-8). Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable the skilled artisan. Exemplary dsRNAs may be synthesized chemically or produced in vitro or in vivo using appropriate expression vectors. Exemplary synthetic RNAs include 21 nucleotide RNAs chemically synthesized using methods known in the art (e.g. Expedite RNA phosphoramidites and thymidine phosphoramidite (Proligo, Germany). Synthetic oligonucleotides are preferably deprotected and gel-purified using methods known in the art (see e.g. Elbashir et al. (2001) Genes Dev. 15: 188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species will be designed to include a portion of nucleic acid sequence represented in a PPIB gene, such as, for example, a nucleic acid that hybridizes, under stringent and/or physiological conditions, to PPIB gene and a complement thereof.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference. Messenger RNA (mRNA) is generally thought of as a linear molecule which contains the information for directing protein synthesis within the sequence of ribonucleotides, however studies have revealed a number of secondary and tertiary structures that exist in most mRNAs. Secondary structure elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g. Jaeger et al. (1989) Proc. Natl. Acad. Sci. USA 86:7706 (1989); and Turner et al. (1988) Annu Rev. Biophys. Biophys. Chem. 17:167). The rules are useful in identification of RNA structural elements and, in particular, for identifying single stranded RNA regions which may represent preferred segments of the mRNA to target for silencing RNAi, ribozyme or antisense technologies. Accordingly, preferred segments of the mRNA target can be identified for design of the RNAi mediating dsRNA oligonucleotides as well as for design of appropriate ribozyme and hammerhead ribozyme compositions of the invention.

The dsRNA oligonucleotides may be introduced into the cell by transfection with an heterologous target gene using carrier compositions such as liposomes, which are known in the art—e.g. Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al. (1998) J Cell Biol 141: 863-74). The effectiveness of the RNAi may be assessed by any of a number of assays following introduction of the dsRNAs. These include Western blot analysis using antibodies which recognize the PPIB gene product following sufficient time for turnover of the endogenous pool after new protein synthesis is repressed, reverse transcriptase polymerase chain reaction and Northern blot analysis to determine the level of existing PPIB target mRNA.

Further compositions, methods and applications of RNAi technology are provided in U.S. Pat. Nos. 6,278,039, 5,723,750 and 5,244,805, which are incorporated herein by reference.

Ribozyme molecules designed to catalytically cleave PPIB mRNA transcripts can also be used to prevent translation of subject PPIB mRNAs and/or expression of PPIB (see, e.g., PCT International Publication WO90/11364, published Oct. 4, 1990; Sarver et al. (1990) Science 247:1222-1225 and U.S. Pat. No. 5,093,246). Ribozymes are enzymatic RNA molecules capable of catalyzing the specific cleavage of RNA. (For a review, see Rossi (1994) Current Biology 4: 469-471). The mechanism of ribozyme action involves sequence specific hybridization of the ribozyme molecule to complementary target RNA, followed by an endonucleolytic cleavage event. The composition of ribozyme molecules preferably includes one or more sequences complementary to a PPIB mRNA, and the well known catalytic sequence responsible for mRNA cleavage or a functionally equivalent sequence (see, e.g., U.S. Pat. No. 5,093,246, which is incorporated herein by reference in its entirety).

In addition to ribozymes that cleave mRNA at site specific recognition sequences, hammerhead ribozymes can also be used to destroy target mRNAs. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art and is described more fully in Haseloff and Gerlach ((1988) Nature 334:585-591; and see PCT Appln. No. WO89/05852, the contents of which are incorporated herein by reference). Hammerhead ribozyme sequences can be embedded in a stable RNA such as a transfer RNA (tRNA) to increase cleavage efficiency in vivo (Perriman et al. (1995) Proc. Natl. Acad. Sci. USA, 92: 6175-79; de Feyter, and Gaudron, Methods in Molecular Biology, Vol. 74, Chapter 43, “Expressing Ribozymes in Plants”, Edited by Turner, P. C, Humana Press Inc., Totowa, N.J.). In particular, RNA polymerase HI-mediated expression of tRNA fusion ribozymes are well known in the art (see Kawasaki et al. (1998) Nature 393: 284-9; Kuwabara et al. (1998) Nature Biotechnol. 16: 961-5; and Kuwabara et al. (1998) Mol. Cell 2: 617-27; Koseki et al. (1999) J Virol 73: 1868-77; Kuwabara et al. (1999) Proc Natl Acad Sci USA 96: 1886-91; Tanabe et al. (2000) Nature 406: 473-4). There are typically a number of potential hammerhead ribozyme cleavage sites within a given target cDNA sequence. Preferably the ribozyme is engineered so that the cleavage recognition site is located near the 5′ end of the target mRNA—to increase efficiency and minimize the intracellular accumulation of non-functional mRNA transcripts. Furthermore, the use of any cleavage recognition site located in the target sequence encoding different portions of the C-terminal amino acid domains of, for example, long and short forms of target would allow the selective targeting of one or the other form of the target, and thus, have a selective effect on one form of the target gene product.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a PPIB mRNA. In addition, ribozymes possess highly specific endoribonuclease activity, which autocatalytically cleaves the target sense mRNA. The present invention extends to ribozymes which hybridize to a sense mRNA encoding a PPIB gene thereby hybridising to the sense mRNA and cleaving it, such that it is no longer capable of being translated to synthesize a functional polypeptide product.

Ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target messages and inhibit translation. Because ribozymes, unlike antisense molecules, are catalytic, a lower intracellular concentration is required for efficiency.

A further aspect of the invention relates to the use of the isolated “antisense” nucleic acids to inhibit expression, e.g., by inhibiting transcription and/or translation of a subject PPIB nucleic acid. The antisense nucleic acids may bind to the potential drug target by conventional base pair complementarity, or, for example, in the case of binding to DNA duplexes, through specific interactions in the major groove of the double helix. In general, these methods refer to the range of techniques generally employed in the art, and include any methods that rely on specific binding to oligonucleotide sequences.

An antisense construct of the present invention can be delivered, for example, as an expression plasmid which, when transcribed in the cell, produces RNA which is complementary to at least a unique portion of the cellular mRNA which encodes a PPIB polypeptide. Alternatively, the antisense construct is an oligonucleotide probe, which is generated ex vivo and which, when introduced into the cell causes inhibition of expression by hybridizing with the mRNA and/or genomic sequences of a PPIB nucleic acid. Such oligonucleotide probes are preferably modified oligonucleotides, which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668.

Antisense approaches involve the design of oligonucleotides (either DNA or RNA) that are complementary to mRNA encoding the PPIB polypeptide. The antisense oligonucleotides will bind to the mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. (Wagner, R. 1994. Nature 372:333). Therefore, oligonucleotides complementary to either the 5′ or 3′ untranslated, non-coding regions of a gene could be used in an antisense approach to inhibit translation of that mRNA. Oligonucleotides complementary to the 5′ untranslated region of the mRNA should include the complement of the AUG start codon. Antisense oligonucleotides complementary to mRNA coding regions are less efficient inhibitors of translation but could also be used in accordance with the invention. Whether designed to hybridize to the 5′, 3′ or coding region of mRNA, antisense nucleic acids should be at least six nucleotides in length, and are preferably less that about 100 and more preferably less than about 50, 25, 17 or 10 nucleotides in length.

The antisense oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or compounds facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytiethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

The antisense oligonucleotide may also comprise at least one modified sugar moiety selected from the group including but not limited to arabinose, 2-fluoroarabinose, xylulose, and hexose. The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

A further aspect of the invention relates to the use of DNA enzymes to inhibit expression of the PPIB gene. DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid. There are currently two basic types of DNA enzymes, and both of these were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions. Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence. When synthesizing the DNA enzyme, the specific antisense recognition sequence that will target the enzyme to the message is divided so that it comprises the two arms of the DNA enzyme, and the DNA enzyme loop is placed between the two specific arms. Methods of making and administering DNA enzymes can be found, for example, in U.S. Pat. No. 6,110,462. Similarly, methods of delivery of DNA ribozymes in vitro or in vivo include methods of delivery of RNA ribozyme, as outlined in detail above. Additionally, one of skill in the art will recognize that, like antisense oligonucleotide, DNA enzymes can be optionally modified to improve stability and improve resistance to degradation.

Antisense RNA and DNA, ribozyme, RNAi constructs of the invention may be prepared by any method known in the art for the synthesis of DNA and RNA molecules, including techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

In some embodiments, the agent is an aptamer. As explained before, aptamers are nucleic acid or peptide molecules that bind to a specific target molecule. Aptamers can inhibit the activity of the target molecule by binding to it.

The term “therapeutically-effective amount” of an agent of this invention means an amount effective to improve the response of the patient to radiation therapy having cancer. Such amounts may comprise from about 0.001 to about 100 mg of the compound per kilogram of body weight of the subject to which the composition is administered. Therapeutically effective amounts can be administered according to any dosing regimen satisfactory to those of ordinary skill in the art.

In some embodiments, the agent is administered to the subject in a pharmaceutical composition. Thus, also provided herein are pharmaceutical compositions containing agents of the invention and a pharmaceutically-acceptable carrier, which are generally accepted in the art for the delivery of biologically active agents to animals, in particular, mammals.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The agent may be administered in the form of a pharmaceutically acceptable salts or prodrugs. The “Pharmaceutically-acceptable salts” refer to derivatives of the disclosed agents or compounds wherein the agent or parent compound is modified by making acid or base salts thereof. Examples of pharmaceutically acceptable salts include, but are not limited to, mineral or organic acid salts of basic residues such as amines, or alkali or organic salts of acidic residues such as carboxylic acids. Pharmaceutically-acceptable salts include the conventional non-toxic salts or the quaternary ammonium salts of the parent compound formed, for example, from non-toxic inorganic or organic acids. Such conventional nontoxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Pharmaceutically acceptable salts are those forms of agents, suitable for use in contact with the tissues of human beings and animals without excessive toxicity, irritation, allergic response, or other problem or complication, commensurate with a reasonable benefit/risk ratio.

Pharmaceutically-acceptable salt forms may be synthesized from the agents which contain a basic or acidic moiety by conventional chemical methods. Generally, such salts are, for example, prepared by reacting the free acid or base forms of these agents with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in at page 1418 of Remington's Pharmaceutical Sciences, 17th ed., Mack Publishing Company, Easton, Pa., 1985.

“Prodrugs” are intended to include any covalently bonded carriers that release an active parent drug or agent of the present invention in vivo when such prodrug is administered to a mammalian subject. Since prodrugs are known to enhance numerous desirable qualities of pharmaceuticals (i.e., solubility, bioavailability, half life, manufacturing, etc.) the agents of the present invention may be delivered in prodrug form. Thus, the present invention is intended to cover prodrugs of the presently claimed compounds, methods of delivering the same, and compositions containing the same. Prodrugs of the present invention are prepared by modifying functional groups present in the agent in such a way that the modifications are cleaved, either in routine manipulation or in vivo, to an active agent. Prodrugs include agents of the present invention wherein an acyl, hydroxy, amino, or sulfhydryl group is bonded to any group that, when the prodrug of the present invention is administered to a mammalian subject, is cleaved to form a free acetyl, hydroxyl, free amino, or free sulfhydryl group, respectively. Examples of prodrugs include, but are not limited to, acetate, formate, and benzoate derivatives of alcohol and amine functional groups in the agents of the present invention.

It will be appreciated by those skilled in the art that some of the agents having a chiral center may exist in, and may be isolated in, optically active and racemic forms. It is to be understood that the term “agent” of the present invention encompasses any racemic, optically-active, regioisomeric or stereoisomeric form, or mixtures thereof, which possess the therapeutically useful properties described herein. It is well known in the art how to prepare optically active forms (for example, by resolution of the racemic form by recrystallization techniques, by synthesis from optically-active starting materials, by chiral synthesis, or by chromatographic separation using a chiral stationary phase). It is also to be understood that the scope of this invention encompasses not only the various isomers, which may exist but also the various mixtures of isomers, which may be formed. For example, if the compound of the present invention contains one or more chiral centers, the compound can be synthesized enantioselectively or a mixture of enantiomers and/or diastereomers can be prepared and separated. The resolution of the compounds of the present invention, their starting materials and/or the intermediates may be carried out by known procedures, e.g., as described in the four volume compendium Optical Resolution Procedures for Chemical Compounds: Optical Resolution Information Center, Manhattan College, Riverdale, N.Y., and in Enantiomers, Racemates and Resolutions, Jean Jacques, Andre Collet and Samuel H. Wilen; John Wiley & Sons, Inc., New York, 1981, which is incorporated in its entirety by this reference. Basically, the resolution of the agents is based on the differences in the physical properties of diastereomers by attachment, either chemically or enzymatically, of an enantiomerically pure moiety resulting in forms that are separable by fractional crystallization, distillation or chromatography.

The agents, including the salts and prodrugs of these agents, of the present invention may be purchased commercially or may also be prepared in ways well known to those skilled in the art of organic synthesis. It is understood by one skilled in the art of organic synthesis that the functionality present on various portions of the molecule must be compatible with the reagents and reactions proposed. Such restrictions to the substituents, which are compatible with the reaction conditions, will be readily apparent to one skilled in the art and alternate methods must then be used.

Pharmaceutically-acceptable carriers are formulated according to a number of factors well within the purview of those of ordinary skill in the art to determine and accommodate. These include, without limitation: the type and nature of the agent; the subject to which the agent-containing composition is to be administered; the intended route of administration of the composition; and, the therapeutic indication being targeted. Pharmaceutically-acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, well known to those of ordinary skill in the art. Descriptions of suitable pharmaceutically-acceptable carriers, and factors involved in their selection, are found in a variety of readily available sources, such as Remington's Pharmaceutical Sciences, 17th ed., Mack Publishing Company, Easton, Pa., 1985.

Administration may be, for example, by various parenteral means. Pharmaceutical compositions suitable for parenteral administration include various aqueous media such as aqueous dextrose and saline solutions; glycol solutions are also useful carriers, and preferably contain a water soluble salt of the active agent, suitable stabilizing compounds, and if necessary, buffering compounds. Antioxidizing compounds, such as sodium bisulfite, sodium sulfite, or ascorbic acid, either alone or in combination, are suitable stabilizing compounds; also used are citric acid and its salts, and EDTA. In addition, parenteral solutions can contain preservatives such as benzalkonium chloride, methyl- or propyl-paraben, and chlorobutanol.

Alternatively, compositions may be administered orally in solid dosage forms, such as capsules, tablets and powders; or in liquid forms such as elixirs, syrups, and/or suspensions. Gelatin capsules can be used to contain the active ingredient and a suitable carrier such as, but not limited to, lactose, starch, magnesium stearate, stearic acid, or cellulose derivatives. Similar diluents can be used to make compressed tablets. Both tablets and capsules can be manufactured as sustained release products to provide for continuous release of medication over a period of time. Compressed tablets can be sugar-coated or film-coated to mask any unpleasant taste, or used to protect the active ingredients from the atmosphere, or to allow selective disintegration of the tablet in the gastrointestinal tract.

A preferred formulation of the invention is a mono-phasic pharmaceutical composition suitable for parenteral or oral administration, consisting essentially of a therapeutically-effective amount of an agent of the invention, and a pharmaceutically acceptable carrier.

Another preferred formulation of the invention is a mono-phasic pharmaceutical composition, consisting essentially of a therapeutically-effective amount of a prodrug of an agent of the invention, and a pharmaceutically acceptable carrier.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as wetting agents, emulsifying agents and dispersing agents. It may also be desirable to include isotonic agents, such as sugars, sodium chloride, and the like in the compositions. In addition, prolonged absorption of the injectable pharmaceutical form may be brought about by the inclusion of agents which delay absorption such as aluminum monosterate and gelatin.

In some cases, in order to prolong the effect of a agent, it is desirable to slow the absorption of the agent from subcutaneous or intramuscular injection. This may be accomplished by the use of a liquid suspension of crystalline or amorphous material having poor water solubility. The rate of absorption of the agent then depends upon its rate of dissolution, which in turn may depend upon crystal size and crystalline form. Alternatively, delayed absorption of a parenterally-administered agent is accomplished by dissolving or suspending the agent in an oil vehicle.

Injectable depot forms are made by forming microencapsulated matrices of the agent in biodegradable polymers such as polylactide-polyglycolide. Depending on the ratio of agent to polymer, and the nature of the particular polymer employed, the rate of agent release can be controlled. Examples of other biodegradable polymers include poly(orthoesters) and poly(anhydrides). Depot injectable formulations are also prepared by entrapping the agent in liposomes or microemulsions which are compatible with body tissue. The injectable materials can be sterilized for example, by filtration through a bacterial-retaining filter.

For preparing solid compositions such as tablets, the principal active ingredient is mixed with a pharmaceutical excipient to form a solid preformulation composition containing a homogeneous mixture of an agent of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the therapeutic compounds of the present invention.

Formulations suitable for oral administration may be in the form of capsules, cachets, pills, tablets, powders, granules or as a solution or a suspension in an aqueous or non-aqueous liquid, or an oil-in-water or water-in-oil liquid emulsions, or as an elixir or syrup, or as pastilles (using an inert base, such as gelatin and glycerin, or sucrose and acacia), and the like, each containing a predetermined amount of an agent of the present invention as an active ingredient. An agent or agents of the present invention may also be administered as bolus, electuary or paste.

In solid dosage forms of the agents for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monosterate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may be employed as fillers in soft and hard-filled gelatin capsules using such excipients as lactose or milk sugars, as well as high molecular weight polyethylene glycols and the like.

A tablet may be made by compression or molding optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter. These compositions may also optionally contain opacifying agents and may be of a composition that they release the active ingredient only, or preferentially, in a certain portion of the gastrointestinal tract, optionally, in a delayed manner. Examples of embedding compositions which can be used include polymeric substances and waxes. The active ingredient can also be in microencapsulated form.

The tablets or pills may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Liquid dosage forms for oral administration of the agents include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or salicylate, and which is solid at room temperature, but liquid at body temperature and, therefore, will melt in the rectum or vaginal cavity and release the active compound. Formulations of the present invention which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches, drops and inhalants. The active ingredient may be mixed under sterile conditions with a pharmaceutically-acceptable carrier, and with any buffers, or propellants which may be required. The ointments, pastes, creams and gels may contain, in addition to an active ingredient, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to an active ingredient, excipients such as lactose, talc, silicic acid, aluminum hydroxide, calcium silicates and polyamide powder or mixtures of these substances. Sprays can additionally contain customary propellants such as chlorofluorohydrocarbons and volatile unsubstituted hydrocarbons, such as butane and propane. Transdermal patches have the added advantage of providing controlled delivery of compounds of the invention to the body. Such dosage forms can be made by dissolving, dispersing or otherwise incorporating one or more agents in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

Pharmaceutical formulations further include those suitable for administration by inhalation or insufflation or for nasal or intraocular administration. For administration to the upper (nasal) or lower respiratory tract by inhalation, the agents may be conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Alternatively, for administration by inhalation or insufflation, the composition may take the form of a dry powder, for example, a powder mix of one or more of the agents and a suitable powder base, such as lactose or starch. The powder composition may be presented in unit dosage form in, for example, capsules or cartridges, or, e.g., gelatin or blister packs from which the powder may be administered with the aid of an inhalator, insufflator or a metered-dose inhaler. For intranasal administration, compounds of the invention may be administered by means of nose drops or a liquid spray, such as by means of a plastic bottle atomizer or metered-dose inhaler. Typical of atomizers are the Mistometer (Wintrop) and Medihaler (Riker). Drops, such as eye drops or nose drops, may be formulated with an aqueous or nonaqueous base also comprising one or more dispersing agents, solubilizing agents or suspending agents. Liquid sprays are conveniently delivered from pressurized packs. Drops can be delivered by means of a simple eye dropper-capped bottle or by means of a plastic bottle adapted to deliver liquid contents dropwise by means of a specially shaped closure.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

The dosage formulations provided by this invention may contain the therapeutic compounds of the invention, either alone or in combination with other therapeutically active ingredients, and pharmaceutically acceptable inert excipients. The dosage formulations may contain one or more of antioxidants, chelating agents, diluents, binders, lubricants/glidants, disintegrants, coloring agents and release modifying polymers.

Suitable antioxidants may be selected from amongst one or more pharmaceutically acceptable antioxidants known in the art. Examples of pharmaceutically acceptable antioxidants include butylated hydroxyanisole (BHA), sodium ascorbate, butylated hydroxytoluene (BHT), sodium sulfite, citric acid, malic acid and ascorbic acid. The antioxidants may be present in the dosage formulations of the present invention at a concentration between about 0.001% to about 5%, by weight, of the dosage formulation.

Suitable chelating agents may be selected from amongst one or more chelating agents known in the art. Examples of suitable chelating agents include disodium edetate (EDTA), edetic acid, citric acid and combinations thereof. The chelating agents may be present in a concentration between about 0.001% and about 5%, by weight, of the dosage formulation.

Suitable diluents such as lactose, sugar, cornstarch, modified cornstarch, mannitol, sorbitol, and/or cellulose derivatives such as wood cellulose and microcrystalline cellulose, typically in an amount within the range of from about 20% to about 80%, by weight.

Examples of suitable binders include methyl cellulose, hydroxypropyl cellulose, hydroxypropylmethyl cellulose, polyvinyl pyrrolidone, eudragits, ethyl cellulose, gelatin, gum arabic, polyvinyl alcohol, pullulan, carbomer, pregelatinized starch, agar, tragacanth, sodium alginate, microcrystalline cellulose and the like.

Examples of suitable disintegrants include sodium starch glycolate, croscarmellose sodium, crospovidone, low substituted hydroxypropyl cellulose, and the like. The concentration may vary from 0.1% to 15%, by weight, of the dosage form.

Examples of lubricants/glidants include colloidal silicon dioxide, stearic acid, magnesium stearate, calcium stearate, talc, hydrogenated castor oil, sucrose esters of fatty acid, microcrystalline wax, yellow beeswax, white beeswax, and the like. The concentration may vary from 0.1% to 15%, by weight, of the dosage form.

Release modifying polymers may be used to form extended release formulations containing the therapeutic compounds of the invention. The release modifying polymers may be either water-soluble polymers, or water insoluble polymers. Examples of water-soluble polymers include polyvinylpyrrolidone, hydroxy propylcellulose, hydroxypropyl methylcellulose, vinyl acetate copolymers, polyethylene oxide, polysaccharides (such as alginate, xanthan gum, etc.), methylcellulose and mixtures thereof. Examples of water-insoluble polymers include acrylates such as methacrylates, acrylic acid copolymers; cellulose derivatives such as ethylcellulose or cellulose acetate; polyethylene, and high molecular weight polyvinyl alcohols.

Optionally, the therapeutic methods of the present invention may be combined with other anti-cancer therapies. Examples of anti-cancer therapies include traditional cancer treatments such as surgery and chemotherapy, as well as other new treatments. Such other anti-cancer therapies will be expected to act in an additive or synergistic manner with the radiation therapy. This may result in better control of the cancer as well as reducing the need for high dosages and reducing any dose related harmful side effects. For example, a wide array of conventional compounds, have been shown to have anti-cancer activities. These compounds have been used as pharmaceutical agents in chemotherapy to shrink solid tumors, prevent metastases and further growth, or decrease the number of malignant cells in leukemic or bone marrow malignancies. Although chemotherapy has been effective in treating various types of malignancies, many anti-cancer compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

Another embodiment of the invention relates to the use of any of the compositions described herein in the preparation of a medicament for improving the response of a cancer patient to radiation therapy.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1

### This Example Illustrates the Development and Evaluation of the Radiation Response Prediction Gene Expression Model (GEM)

A schematic of model generation and validation process is depicted in FIG. 6 and is described in detail below.

Briefly, radiosensitivity data for both the bladder cancer (BLA-40, Table 1A) and primary Human Skin Fibroblasts (HSF) developed from skin biopsies collected from areas outside of the radiation field in patients undergoing radiotherapy (Brock, Table 1A), in the form of SF2 are shown in Tables 2A and 2B, respectively. Of the 8470 Affymetrix HG-U133A probe sets that had matching Illumina probes, the inventors found that 7515 probe sets survived the COXEN coexpression step between bladder cell lines and human tumors. The 300 probe sets most differentially expressed between the 17 most radiosensitive (SF2 range 0.19-0.51, avg. 0.39) and 10 most radioresistant (SF2 range 0.72-0.98, avg. 0.86) cell lines were chosen as candidate biomarkers for inclusion in the radiation response GEM.

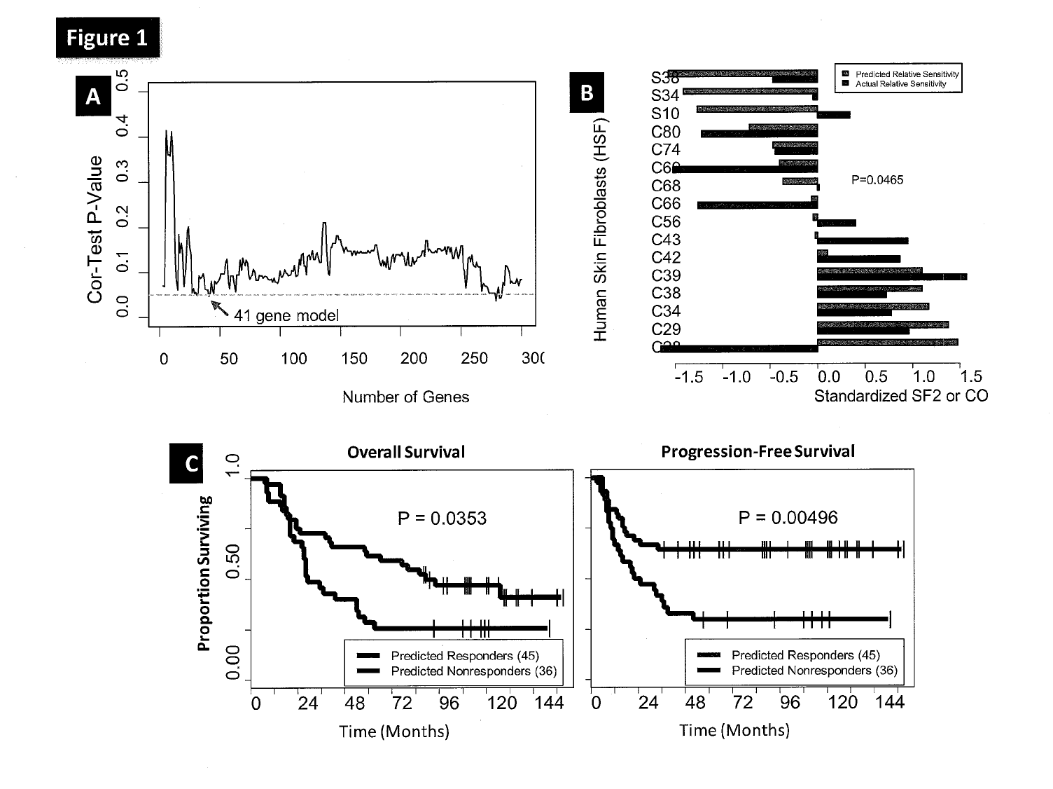

The inventors then assessed how well GEMs, constructed reiteratively from these candidate biomarkers as described below, were able to predict radiation sensitivity in the HSF cell line panel and selected a 41-probeset model (Table 3) that was best able to predict radiosensitivity in this panel (FIG. 1A). A comparison of the predicted and true radiosensitivity data for the HSF panel using the 41 gene GEM is shown in FIG. 1B.

### Development of the Radiation Response Prediction Gene Expression Model (GEM)

The inventors used COXEN to develop a model predictive of radiation response. FIG. 6A shows a schematic depiction of the methodology. The inventors used the BLA-40 bladder cancer cell line dataset (Table 1A) to discover genes differentially expressed according to radiosensitivity. To determine the subset of these genes whose expression is relevant to human primary tumors the inventors determined which of these shared similar patterns of co-expression with the human bladder tumor sample dataset (Table 1A). For each of the remaining probe sets, the inventors measured the significance of differential expression between the 17 most radiosensitive (SF2 range 0.19-0.51, avg. 0.39) and 10 most radioresistant (SF2 range 0.72-0.98, avg. 0.86) cell lines using Student's t-test. These numbers were chosen to maximize the number of genes differentially expressed as a function of intrinsic sensitivity to radiation as measured by SF2. The inventors selected the 300 most significantly differentially expressed probe sets as candidate biomarkers for radiation response prediction.

The inventors used a linear discriminant analysis approach (LDA) to develop the optimal gene model predictors of radiation response from the 300 probes above as described in detail below. To select a GEM that effectively predicts radiation response of several cancer types, the inventors tested the performance of candidate GEMs at predicting radiation sensitivity within the Brock cell panel (Table 1B), as measured by the correlation between predicted sensitivities and actual SF2 values. Candidate GEMs were iteratively constructed from the 300 candidate probe sets above, starting with a model consisting of the top three candidate biomarkers and then successively adding biomarkers until all candidate biomarkers were used. GEMs that resulted in a higher magnitude of correlation between predicted and actual sensitivity were more accurate at response prediction. The inventors selected a GEM that had the highest correlation with the smallest number of probes.

Patient Datasets

As part of a study to develop predictors of metastasis among head and neck squamous-cell cancer (HNSCC) patients, 81 tumor samples were transcriptionally profiled on Affymetrix HG-U133 Plus 2.0 GeneChips, and this information is publicly available on ArrayExpress (arrayexpress accession number E-TABM-302). The clinical characteristics of the patients in this dataset are summarized in Table 1B. Importantly, 73 of these patients were treated only with radiation therapy.

A third set was the result of the NCI Director's Challenge “Toward a Molecular Classification of Lung Adenocarcinoma” project where several hundred lung adenocarcinoma tumors have been transcriptionally profiled on Affymetrix HG-U133A GeneChips. Expression profiles for these tumor samples, in addition to clinical characteristics including relapse-free and overall survival time, are publicly available at the NCI web site. The clinical characteristics of the patients in this dataset are summarized in Table 1C. Of these samples, only 65 were treated with radiotherapy; twenty of these were treated only with radiation therapy.

Additional validation was carried out on a set comprised of 118 patients with head and neck squamous cell carcinoma (HNSCC) treated between 2002 and 2008 at the University of Virginia with definitive radiation therapy alone or in combination with induction and/or concurrent chemotherapy regimens. Patients with T1-T2 primary tumors and NO-1 nodal disease were treated with radiation alone and patients with T3-4 primary tumors or N2-3 nodal disease received radiation and chemotherapy. Archived pre-treatment primary tumor biopsies were available for 72 patients and were used to create a tissue microarray. Survival was calculated from the date of diagnosis to the date of death of any cause or last date of follow-up. The clinical characteristics of the patients in this dataset are published (Shonka D C, Jr., Shoushtari A N, Thomas C Y, Moskaluk C, Read P W, Reibel J F, Levine P A, and Jameson M J. Predicting residual neck disease in patients with oropharyngeal squamous cell carcinoma treated with radiation therapy: utility of p16 status. Arch Otolaryngol Head Neck Surg. 2009; 135:1126-1132).

Cell Line Datasets

The datasets used in this study are listed in Table 1A. The bladder cancer cell line dataset was used to select biomarkers and train the prediction model. The gene expression profiles of unirradiated samples of these cell lines have previously been measured using Affymetrix HG-U133A GeneChips. The inventors used a human bladder tumor sample dataset to find genes concordantly regulated in these bladder cell lines and human tumors. This dataset consisted of 60 expression profiles downloaded from the Gene Expression Omnibus (GEO) web site (GEO Accession Number GSE3167), as well as 25 profiles obtained locally. The gene expression profile and radiation sensitivity of a panel of 16 primary human skin fibroblast (HSF) cell lines developed from skin biopsies (Brock) from radiotherapy patients (collected from areas outside of the radiation field) was used to test prediction results derived from the bladder cell lines and human data and to refine model selection. These 16 cell lines were hybridized to Illumina WholeGenome6 v2 and v3 chips. Samples of these cell lines were exposed to a total absorbed dose of 2 Gy of ionizing radiation, after which the survival fraction was measured.

Gene Array Data Processing

Affymetrix datasets were background adjusted, quantile normalized, and summarized using the Robust Multichip Average technique. Illumina data were processed according to the manufacturer. As Table 1 indicates, gene expression profiles were measured using several different microarray platforms. In order to generate a consistent prediction model that could be applied to any of these datasets, the inventors limited subsequent analyses to genes that were present on all platforms. First, as all 22215 Affymetrix HG-U133A probe sets are also represented among the 54613 probe sets present on the Affymetrix HG-U133 Plus 2.0 chip, the inventors kept the expression values for this subset in the head and neck human tumor dataset and discarded the rest. Second, as the head and neck cell line dataset was profiled using two versions of the Illumina WholeGenome-6 chip, the inventors used a file downloaded from the Illumina website (illumina.com) to map identical or closely matching probes between the two versions. Most of the cell lines in this experiment were profiled using version 2 of the WG6 chip, which has 48701 probes. The cell lines C42, S34, and S38 were profiled using version 3, which has 48803 probes. There are 43071 probes that are identical or closely matching in the two versions. Affymetrix probe sets were mapped to Illumina probes using a file downloaded from the Illumina web site (illumina.com), which indicated the probe sets and probes that corresponded to the same RefSeq identifier. With these matching steps completed, 8406 unique Illumina probes corresponded to 8470 unique Affymetrix Probe Sets.

Linear Discriminant Analysis

The inventors used a linear discriminant analysis approach (LDA) to develop the optimal gene model predictors of radiation response from the 300 probes above. In general, for any given gene expression model (GEM) which consists of a list of probe sets, the inventors train a linear discriminant using the expression values of the probe sets in the model for the radiation-sensitive and radiation-resistant bladder cancer cell lines. The inventors apply this discriminant on the expression values of an “optimization” cell line or patient dataset to classify the sample as a responder or non-responder. Each sample receives a GEM score, which represents the posterior probability that the sample is sensitive to radiation.

### Example 2

### Illustrates the Accuracy and Specificity of the Radiation Response Prediction GEM in Patient Datasets

Utility of the 41 gene GEM in stratifying clinical outcome of patients treated with radiotherapy.

The inventors used the 41 gene GEM to predict the clinical response of patients with either lung cancer or HNSCC enrolled in two independent clinical studies. The Rickman HNSCC patient dataset (Tables 1A and 1B) comprised 81 patients, of which 73 were treated using radiotherapy alone, whereas 8 patients were treated with an unspecified chemotherapeutic regimen in addition to radiotherapy. Since chemotherapy may have a significant influence on patient response, and because the subset of patients treated with radiotherapy alone was sufficiently large, the inventors restricted the prediction assessment analysis to these 73 patients. Each patient was first assigned a GEM score indicating the predicted relative probability of response to radiation. To assess prediction performance the inventors generated an ROC curve which had a Wilcoxon Rank-Sum test p-value of 0.015, indicating that the predictor was significantly better than random, and an area under the curve (AUC) of 0.61. This ROC curve was used to select a GEM score threshold value that allowed stratification of this group into predicted responders and predicted non-responders. Kaplan-Meier analysis revealed significant separation in survival time between the predicted responders and non-responders in overall and progression-free survival (FIG. 1C).

Since in addition to radiation response, other patient and tumor characteristics influence patient survival, the inventors used Cox proportional hazards analysis to determine the contribution of the GEM response scores in addition to these other characteristics. All patients with complete clinical information (72 of 73) and all available covariates were included in this analysis with the stepwise AIC model selection discarding the variables least significant to the endpoint. Results shown in Table 4A (for overall survival) and Table 4B (for progression-free survival) indicate the GEM score is a significant variable predicting the overall survival hazard rate (p=0.047). Table 4B shows that no variable relates to progression-free survival hazard rate, but the GEM score has the lowest p-value (p=0.068).

Next, the inventors evaluated GEM score outcome prediction in patients with lung adenocarcinoma in the Shedden dataset. A complicating factor for this dataset is the fact that a majority (45 of 65) of the patients received an unspecified chemotherapeutic regimen in addition to radiotherapy, limiting us to multivariate analysis. Cox proportional hazards analysis involving all variables, for all patients with complete information (63 of 65), shows that treatment with chemotherapy, lymph node metastasis status, and GEM score all significantly affect the hazard rate (Table 4C). Therefore, the inventors performed Cox proportional hazards analyses on 20 patients who received only radiotherapy. Both nodal status and GEM score were borderline significant for the prediction of overall survival hazard rate for this set of patients (Table 4D).

### Predictive Performance of Published Gene Expression Models in Lung and HNSCC Patients

The inventors also sought to determine if two recently published predictive models of radiation response in patients could predict outcome in the datasets. Importantly, neither model predicted clinical outcome in the Shedden or Rickman datasets.

The inventors evaluated whether the Weichselbaum et al. (Weichselbaum R R, Ishwaran H, Yoon T, Nuyten D S, Baker S W, Khodarev N, Su A W, Shaikh A Y, Roach P, Kreike B, et al. An interferon-related gene signature for DNA damage resistance is a predictive marker for chemotherapy and radiation for breast cancer. Proc Natl Acad Sci U.S.A. 2008; 105: 18490-18495) IFN-related DNA damage resistance signature (IRDS) found to predict efficacy of adjuvant chemo-radiotherapy in breast cancer patients, was predictive in the samples. The inventors applied the IRDS model to the head and neck squamous cell carcinoma and lung adenocarcinoma datasets described above and this did not successfully stratify patients according to response in either ROC (Rickman dataset, Wilcoxon Rank-Sum test p-value=0.5; Shedden dataset, Wilcoxon Rank-Sum test p-value=0.627) or Kaplan-Meier (evaluating multiple points on ROC as dichotomizing cutoffs) analyses (Rickman dataset, best overall survival Kaplan-Meier χ² p-value=0.131, best progression-free survival Kaplan-Meier χ² p-value=0.258; Shedden dataset, best overall survival Kaplan-Meier χ² p-value=0.775, best relapse-free survival Kaplan-Meier χ² p-value=0.395) or multivariate COX-proportional hazards analyses (Rickman dataset, overall survival p-value=0.470, progression-free survival p-value=0.61; Shedden dataset, overall survival p-value=0.79, relapse-free survival p-value=0.34).

The second model was one describing a 10 gene radiosensitivity index (RSI, high index=radioresistance) [Eschrich S A, Pramana J, Zhang H, Zhao H, Boulware D, Lee J H, Bloom G, Rocha-Lima C, Kelley S, Calvin D P, et al. A gene expression model of intrinsic tumor radiosensitivity: prediction of response and prognosis after chemoradiation. Int J Radiat Oncol Biol Phys. 2009; 75: 489-496]. The inventors evaluated this RSI model on the BLA-40, Rickman and Shedden datasets. RSI was not significantly correlated with SF2 in the BLA-40 cell lines (pearson correlation=0.183, one-sided cor.test p=0.132, spearman correlation=0.174, one-sided cor.test p=0.144). For the Rickman dataset, RSI was not significantly different between survivors and deceased or between patients who relapsed and patients who did not relapse, for the entire set of patients treated with radiation (with or without chemotherapy) or for the set of patients treated with radiation alone. For the Shedden dataset, the RSI was not significantly different between relapsed and non-relapsed patients (for the entire set of 65 patients who received radiation therapy (with or without chemotherapy), the set of patients who received radiation and chemotherapy (N=45) and the set of patients who received radiation alone, N=20). However, the difference in RSI between survivors and deceased for the entire dataset (N=364+65) approached significance (1-sided p=0.079) and the difference in the dataset of patients who received radiation (N=65) was significant (1-sided p=0.04). However, the RSI was higher in the living patients, which is surprising since RSI is supposed to be directly proportional to radiation resistance. Taken together, this strongly suggests that there is no published model that predicts outcome following radiation for lung and head and neck tumor types.

### Specificity of the 41 Gene GEM in Stratifying Outcome of Lung Cancer Patients

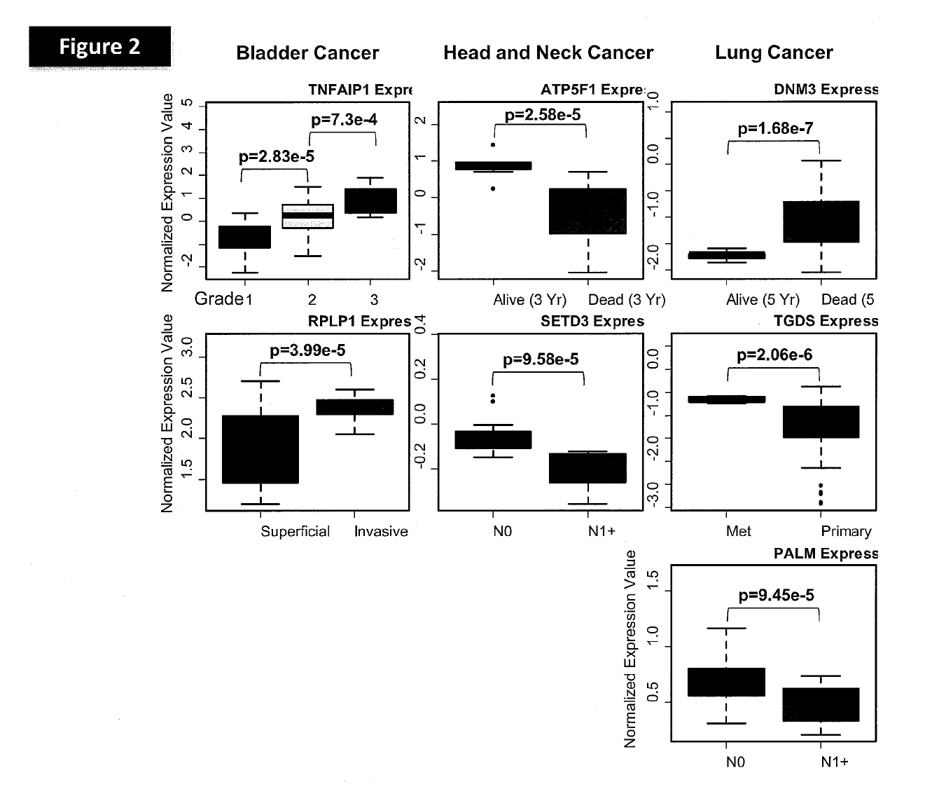

To evaluate the specificity of the 41 probe GEM in predicting clinical outcome after radiation, the inventors used multivariate analysis as described above to evaluate the ability of the model to stratify outcome of 364 lung cancer patients from the same dataset but who were not treated with radiation therapy. In multivariate analysis using all available clinical parameters gender, age, race, N stage, T stage, grade, administration of chemotherapy, and the GEM score, the GEM score did not survive the stepwise AIC variable selection as a predictor for either progression-free or overall survival (p>0.05). Further supporting the notion that the 41 gene GEM informs about clinical outcome after radiation and not general tumor aggressiveness in bladder, lung or HNSCC cancer is the finding that only 7 of 41 genes are associated with stage, grade, invasive ability or clinical outcome in independently profiled cancer datasets found in Oncomine (FIG. 2). Together, these two analyses suggest the 41 gene GEM informs about clinical outcome after radiation rather than a general reflection of tumor aggressiveness.

### Example 3

### This Example Illustrates the Characteristics and Network Analysis of the 41 Genes in the GEM

To explore the functional properties of the genes in the radiotherapy response prediction GEM, the inventors found the gene information corresponding to the probe sets from the NetAffx website. The inventors queried the PANTHER Classification System database at pantherdb.org for gene ontology information corresponding to the genes in the model. No molecular function classification (FIG. 6A) was found for 15 of the 41 genes, while no biological process classification (FIG. 6B) was found for 16 of the 41 genes in the model. No GO terms were significantly over-represented among the genes for which such terms are available (hypergeometric test). However, biological process terms that are represented multiple times included immunity and defense (5 genes), transport (4 genes), cell proliferation and differentiation (3 genes), induction of apoptosis (3 genes). Gene expression and protein synthesis, activation, and destruction terms are also represented multiple times: mRNA transcription regulation (2 genes), protein biosynthesis (3 genes), protein folding (2 genes), protein phosphorylation (3 genes), and proteolysis (4 genes). The most common GO Biological Process classes were unclassified, transport, immunity and defense and proteolysis while the GO Molecular Function classes were unclassified, nucleic acid binding, ribosomal proteins and transcription factors.

The inventors used the Ingenuity Pathways Analysis program (Ingenuity® Systems, ingenuity.com) to generate networks that include genes in the radiation response GEM. The inventors also searched the Oncomine database to determine which genes from the radiation response GEM were associated with tumor stage, grade and outcome in bladder, HNSCC and lung cancer.

Unsupervised analysis revealed the top scoring network comprised 39 of the 41 genes of the GEM. In this network, the 39 genes of the radiation response model were found to interact with genes such as KRAS, HRAS, MYC, MYCN, ABL1, ERBB2, PIK3R1, P38 MAPK, NFkB, and ERK that are known to be involved in radiation response. These findings suggested genes in the model may also have causal roles in radioresistance.

The datasets searched for associations of 41 gene GEM and tumor parameters and clinical outcome can be found at: Blayeri, E., Simko, J. P., Korkola, J. E., Brewer, J. L., Baehner, F., Mehta, K., et al. (2005). Bladder cancer outcome and subtype classification by gene expression. Clinical Cancer Research, 11(11), 4044-55. doi: 10.1158/1078-0432.CCR-04-2409; Chen, H., Yu, S., Chen, C., Chang, G., Chen, C., Yuan, A., et al. (2007). A five-gene signature and clinical outcome in non-small-cell lung cancer. The New England Journal of Medicine, 356(1), 11-20. doi: 10.1056/NEJMoa060096; Cromer, A., Carles, A., Millon, R., Ganguli, G., Channel, F., Lemaire, F., et al. (2004). Identification of genes associated with tumorigenesis and metastatic potential of hypopharyngeal cancer by microarray analysis. Oncogene, 23(14), 2484-98. doi: 10.1038/sj.one.1207345; Ding, L., Getz, G., Wheeler, D. A., Mardis, E. R., McLellan, M. D., Cibulskis, K., et al. (2008). Somatic mutations affect key pathways in lung adenocarcinoma. Nature, 455(7216), 1069-75. doi: 10.1038/nature07423; Dyrskjot, L., Kruhoffer, M., Thykjaer, T., Marcussen, N., Jensen, J. L., Moller, K., et al. (2004). Gene expression in the urinary bladder: a common carcinoma in situ gene expression signature exists disregarding histopathological classification. Cancer Res, 64, 4040-4048; Dyrskjot, L., Zieger, K., Real, F. X., Malats, N., Carrato, A., Hurst, C., et al. (2007). Gene expression signatures predict outcome in non-Muscle-Invasive bladder carcinoma: a multicenter validation study. Clinical Cancer Research, 13(12), 3545-3551. doi: 10.1158/1078-0432.CCR-06-2940; Dyrskjot, L., Zieger, K., Kruhoffer, M., Thykjaer, T., Jensen, J. L., Primdahl, H., et al. (2005). A molecular signature in superficial bladder carcinoma predicts clinical outcome. Clinical Cancer Research 11(11), 4029-36. doi: 10.1158/1078-0432.CCR-04-2095; Frierson, H. F., El-Naggar, A. K., Welsh, J. B., Sapinoso, L. M., Su, A. I., Cheng, J., et al. (2002). Large scale molecular analysis identifies genes with altered expression in salivary adenoid cystic carcinoma. The American journal of pathology, 161(4), 1315-23; Garber, M. E., Troyanskaya, O. G., Schluens, K., Petersen, S., Thaesler, Z., Pacyna-Gengelbach, M., et al. (2001). Diversity of gene expression in adenocarcinoma of the lung. Proceedings of the National Academy of Sciences USA, 98(24), 13784-9. doi: 10.1073/pnas.241500798; Ginos, M. A., Page, G. P., Michalowicz, B. S., Patel, K. J., Volker, S. E., Pambuccian, S. E., et al. (2004). Identification of a gene expression signature associated with recurrent disease in squamous cell carcinoma of the head and neck. Cancer Research, 64(1), 55-63; Hensen, E. F., De Herdt, M. J., Goeman, J. J., Oosting, J., Smit, V. T., Cornelisse, C. J., et al. (2008). Gene-expression of metastasized versus non-metastasized primary head and neck squamous cell carcinomas: a pathway-based analysis. BMC cancer, 8, 168. doi: 10.1186/1471-2407-8-168; Huang, Y., Prasad, M., Lemon, W. J., Hampel, H., Wright, F. A., Kornacker, K., et al. (2001). Gene expression in papillary thyroid carcinoma reveals highly consistent profiles. Proceedings of the National Academy of Sciences USA, 98(26), 15044-9. doi: 10.1073/pnas.251547398; Jain, S., Watson, M. A., DeBenedetti, M. K., Hiraki, Y., Moley, J. F., Milbrandt, J., et al. (2004). Expression profiles provide insights into early malignant potential and skeletal abnormalities in multiple endocrine neoplasia type 2B syndrome tumors. Cancer Research, 64(11), 3907-13. doi: 10.1158/0008-5472.CAN-03-3801; Kuriakose, M. A., Chen, W. T., He, Z. M., Sikora, A. G., Zhang, P., Zhang, Z. Y., et al. (2004). Selection and validation of differentially expressed genes in head and neck cancer. Cellular and molecular life sciences: CMLS, 61(11), 1372-83. doi: 10.1007/s00018-004-4069-0; Larsen, J. E., Pavey, S. J., Passmore, L. H., Bowman, R., Clarke, B. E., Hayward, N. K., et al. (2007). Expression profiling defines a recurrence signature in lung squamous cell carcinoma. Carcinogenesis, 28(3), 760-6. doi: 10.1093/carcin/bgl207; Landi, M. T., Dracheva, T., Rotunno, M., Figueroa, J. D., Liu, H., Dasgupta, A., et al. (2008). Gene expression signature of cigarette smoking and its role in lung adenocarcinoma development and survival. (D. Albertson) PLoS ONE, 3(2), e1651. Vienna: Network Theory. doi: 10.1371/journal.pone.0001651; Modlich, O., Prisack, H., Pitschke, G., Ramp, U., Ackermann, R., Bojar, H., et al. (2004). Identifying superficial, muscle-invasive, and metastasizing transitional cell carcinoma of the bladder: use of cDNA array analysis of gene expression profiles. Clinical Cancer Research, 10(10), 3410-21. doi: 10.1158/1078-0432.CCR-03-0134; O'Donnell, R. K., Kupferman, M., Wei, S. J., Singhal, S., Weber, R., O'Malley, B., et al. (2005). Gene expression signature predicts lymphatic metastasis in squamous cell carcinoma of the oral cavity. Oncogene, 24(7), 1244-51. doi: 10.1038/sj.one.1208285; Raponi, M., Zhang, Y., Yu, J., Chen, G., Lee, G., Taylor, J. M., et al. (2006). Gene expression signatures for predicting prognosis of squamous cell and adenocarcinomas of the lung. Cancer research, 66(15), 7466-72. doi: 10.1158/0008-5472.CAN-06-1191; Roepman, P., Wessels, L. F., Kettelarij, N., Kemmeren, P., Miles, A. J., Lijnzaad, P., et al. (2005). An expression profile for diagnosis of lymph node metastases from primary head and neck squamous cell carcinomas. Nature genetics, 37(2), 182-6. doi: 10.1038/ng1502; Rohrbeck, A., Neukirchen, J., Rosskopf, M., Pardillos, G. G., Geddert, H., Schwalen, A., et al. (2008). Gene expression profiling for molecular distinction and characterization of laser captured primary lung cancers. Journal of Translational Medicine, 6, 69. doi: 10.1186/1479-5876-6-69; Sanchez-Carbayo, M., Lozano, J., Socci, N. D., Saint, F., & Cordon-Cardo, C. (2006). Defining molecular profiles of poor outcome in patients with invasive bladder cancer using oligonucleotide microarrays. Journal of Clinical Oncology, 24(5), 778-89. doi: 10.1200/JCO.2005.03.2375; Schlingemann, J., Habtemichael, N., Ittrich, C., Toedt, G., Kramer, H., Hambek, M., et al. (2005). Patient-based cross-platform comparison of oligonucleotide microarray expression profiles. Laboratory Investigation, 85(8), 1024-39. doi: 10.1038/labinvest.3700293; Shedden, K., Taylor, J. M., Enkemann, S. A., Tsao, M., Yeatman, T. J., Gerald, W. L., et al. (2008). Gene expression-based survival prediction in lung adenocarcinoma: a multi-Site, blinded validation study. Nature Medicine, 14(8), 822-827. doi: 10.1038/nm.1790; Slebos, R. J., Yi, Y., Ely, K., Carter, J., Evjen, A., Zhang, X., et al. (2006). Gene expression differences associated with human papillomavirus status in head and neck squamous cell carcinoma. Clinical Cancer Research, 12(3 Pt 1), 701-9. doi: 10.1158/1078-0432.CCR-05-2017; Stearman, R. S., Dwyer-Nield, L., Zerbe, L., Blaine, S. A., Chan, Z., Bunn, P. A., et al. (2005). Analysis of orthologous gene expression between human pulmonary adenocarcinoma and a carcinogen-induced murine model. The American journal of pathology, 167(6), 1763-75; Stransky, N., Vallot, C., Reyal, F., Bernard-Pierrot, I., de Medina, S. G., Segraves, R., et al. (2006). Regional copy number-independent deregulation of transcription in cancer. Nature Genetics, 38(12), 1386-96. doi: 10.1038/ng1923; Su, L., Chang, C., Wu, Y., Chen, K., Lin, C., Liang, S., et al. (2007). Selection of DDX5 as a novel internal control for Q-RT-PCR from microarray data using a block bootstrap re-sampling scheme. BMC genomics, 8, 140. doi: 10.1186/1471-2164-8-140; Talbot, S. G., Estilo, C., Maghami, E., Sarkaria, I. S., Pham, D. K., O-charoenrat, P., et al. (2005). Gene expression profiling allows distinction between primary and metastatic squamous cell carcinomas in the lung. Cancer Research, 65(8), 3063-71. doi: 10.1158/0008-5472.CAN-04-1985; Tomida, S., Koshikawa, K., Yatabe, Y., Harano, T., Ogura, N., Mitsudomi, T., et al. (2004). Gene expression-based, individualized outcome prediction for surgically treated lung cancer patients. Oncogene, 23(31), 5360-70. doi: 10.1038/sj.one.1207697; Toruner, G. A., Ulger, C., Alkan, M., Galante, A. T., Rinaggio, J., Wilk, R., et al. (2004). Association between gene expression profile and tumor invasion in oral squamous cell carcinoma. Cancer genetics and cytogenetics, 154(1), 27-35. doi: 10.1016/j.cancergencyto.2004.01.026; Wachi, S., Yoneda, K., & Wu, R. (2005). Interactome-transcriptome analysis reveals the high centrality of genes differentially expressed in lung cancer tissues. Bioinformatics, 21(23), 4205-8. doi: 10.1093/bioinformatics/bti688; Wigle, D. A., Jurisica, I., Radulovich, N., Pintilie, M., Rossant, J., Liu, N., et al. (2002). Molecular profiling of non-small cell lung cancer and correlation with disease-free survival. Cancer Research, 62(11), 3005-8; Yamagata, N., Shyr, Y., Yanagisawa, K., Edgerton, M., Dang, T. P., Gonzalez, A., et al. (2003). A training-testing approach to the molecular classification of resected non-small cell lung cancer. Clinical Cancer Research, 9(13), 4695-704.

### Example 4

### This Example Illustrates that Expression of Cyclophilin B Correlates with Radiosensitivity In Vitro

To determine which of the 39 genes in this network have causal roles in radioresistance, the inventors identified those whose expression was most strongly and directly related with this phenotype in a third cell panel, the NCI60 (Table 1A).

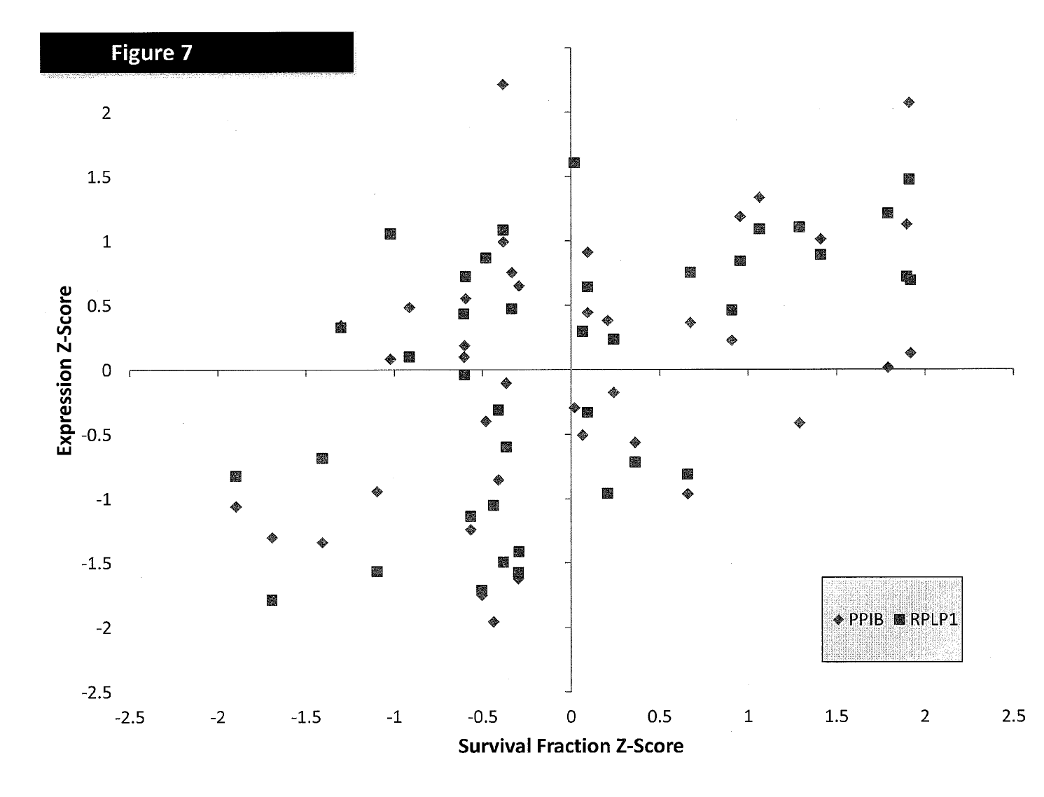

The top 2 genes with the strongest correlation of expression to radioresistance were Cyclophilin B (PPIB), and Acidic Ribosomal Phosphoprotein P1 (RPLP1) (FIG. 7). Given this finding, the inventors sought to evaluate if expression of these genes regulated this phenotype. Use of siRNA provided depletion of both proteins in UMUC-13d bladder cancer cells (FIG. 3A). Reduced levels of PPIB and RPLP1 were associated with reduction in cell number following depletion (FIG. 3A) and this was due to enhanced apoptosis (FIG. 3B). When 6 human cancer cell lines were transiently depleted of either PPIB or RPLP1 and irradiated the inventors noted that cells with reduced levels of either PPIB or RPLP1 had reduced clonogenicity (FIG. 3C).

Since cyclophilins are bound and inhibited by cyclosporine A (CsA), the inventors sought to evaluate whether cyclosporine A recapitulated the observed reduction in cell number observed with PPIB depletion, the inventors carried out a dose response in UMUC-13d bladder cancer cells. FIG. 4A indicates cyclosporin A can diminish overall cell numbers over a 48 hour period compared to vehicle treated cells yet this effect occurs only with doses >8 uM. Interestingly, transient depletion of either PPIB or RPLP1 with and without CsA in UMUC-13d cells indicated that only depletion of RPLP1 with CsA addition results in enhanced apoptosis compared to all other experimental groups suggesting functional equivalence of PPIB and CsA in regard to this phenotype (FIG. 4B). The inventors next examined the effect of PPIB depletion with and without CsA addition on the in vitro clonogenic ability of UMUC-13d cells following exposure to radiation and found that PPIB depletion or CsA had similar effects while PPIB depletion combined with CsA did not result in further reduction in clonogenic potential (FIG. 4C). Finally, the data reveals that both PPIB depletion and CsA inhibit DNA repair and their combined use does not reduce this further suggesting these act on the same pathway(s) regulating DNA repair (FIG. 4D). This result mirrors the findings on clonogenicity (FIG. 4C).

Cyclophilin B and p16 Immunohistochemistry (IHC)

For the Virginia head and neck squamous cell carcinoma patient dataset, four-micron histologic sections were cut, placed on charged glass slides (Superfrost Plus, Fisher Scientific, Pittsburgh, Pa.), deparaffinized in xylene and rehydrated in a graded series of ethanol baths. The sections were immersed in Target Retrieval Solution, Citrate pH 6.0 (Dako, Glostrup, Denmark), and antigen retrieval performed in a Pascal Pressure Chamber (Dako), achieving 22 psi pressure for 30 seconds at 125° C. Immunohistochemistry was performed on a robotic platform (Autostainer, Dako). Endogenous peroxidases were blocked using Peroxidase and Alkaline Phosphatase Blocking Reagent (Dako). Polyclonal rabbit antibody to Cyclophilin B (Cat.#Ab16045, Abcam) was diluted 1:400, and mouse

Cell Line Irradiation, Clonogenic Survival Assay, and Estimation of Radiosensitivity and DNA Repair

Thirty-nine bladder cancer cell lines from a previously described panel of 40 (BLA-40) were cultured and irradiated with a total dose of 2 Gy, and the fraction surviving was determined (SF2) as described. In a similar fashion, using a panel of 16 primary HSF cell lines developed from skin biopsies (Brock) from radiotherapy patients (collected from areas outside the radiation field), survival curves were generated and SF2 was calculated from those curves using a linear quadratic fit (α/β). Preradiation RNA from these 16 HSF cell lines was hybridized to Illumina WholeGenome6 v2 and v3 chips. Exponentially growing cells were transfected with siRNA and/or treated with CsA as described in figures legends. After the treatments, cells were irradiated at ambient temperature with 2 and 6 Gy of x-ray (250 keV) and replated into 100-mm-diameter culture dishes at densities calculated to yield 50 to 100 cell colonies per dish. After 10 to 14 days of incubation, cells were fixed and stained with crystal violet in 20% ethanol, and colonies more than 50 cells were counted. The number of surviving colonies divided by the number of plated cells was used to calculate the plating efficiency and survival fraction for each treatment.

Analysis of DNA Damage Repair by the Comet Assay

Analysis of DNA damage repair by the comet assay was carried out as described. The inventors used the standard comet assay (Trevigen) to Q7 compare the differences in DNA damage repair between wild-type and siRNA knockdown cells. Briefly, exponentially growing cells were irradiated (x-ray, 10 Gy) and allowed to recover for 1 hour. Cells were harvested, mixed with low-melting agarose, and applied to comet slides. After lysis and alkaline unwinding, the electrophoresis was performed under alkaline (pH >13) denaturating conditions at 1 V/cm for 30 minutes. Slides were stained with SYBR green dye for 10 minutes. One hundred randomly selected cells per sample were captured under a fluorescent microscope and analyzed. The relative length and intensity of SYBR green-stained DNA tails to heads were proportional to the amount of DNA damage present in the individual nuclei and were measured by the Olive tail moment. Cyclosporine was purchased from Sigma (St Louis, Mo.) and used in vitro as described.

In Vitro Cell Growth

Bladder cancer cells were seeded in 96-well cell culture plates at a density of 5000 per well in a volume of 200 μl. Twenty-four hours later, the cells were transfected with the siRNA duplexes described previously (6.25 nM) using Oligofectamine according to the manufacturer's instructions in triplicate. Twenty-four hours later, the cells were treated either with indicated concentrations of CsA or with equal volumes of carrier (100% ethanol) in triplicate. Plates were incubated for 24 to 48 hours with carrier or drug. The cell numbers were assessed by alamarBlue (Invitrogen, Carlsbad, Calif.) per the manufacturer's instructions.

Apoptosis Assay

Bladder cancer cells were seeded in six-well cell culture plates at a density of 83,000 per well in a volume of 2 ml. Twenty-four hours later, the cells were transfected with the siRNA duplexes described previously (6.25 nM) using Oligofectamine (Invitrogen) according to the manufacturer's instructions. Apoptosis was assessed by the Annexin V-FITC Apoptosis Detection Kit I (BD Biosciences, Franklin Lakes, N.J.) per the manufacturer's instructions.

Small Interfering RNAs

The following siRNA duplexes were chemically synthesized; Red Fluorescent Protein Ctrl siRNA duplex served as a transfection efficiency control and as a negative control. Luciferase (GL2) siRNA duplex served as a negative control. deprotected and annealed by Dharmacon (Lafayette, Colo.): Cyclophilin B (PPIB) siRNA duplex.

Ribosomal protein, large, P1 (RPLP1) siRNA duplex was chemically synthesized, deprotected and annealed by Sigma-Proligo (St. Louis, Mo.), catalog number SASI_Hs01_{—}00160252. Bladder cancer cells were grown at 50% confluence in 100 mm plates and six-well plates were transfected with the siRNA duplexes (6.25 nmol/L) using Oligofectamine™ (Invitrogen, Carlsbad, Calif.) according to the manufacturer's instructions.

Western Blotting

Western blotting was performed as detailed previously. Antibodies against PPIB (clone k2e2, Santa Cruz Biotechnology, Inc., Santa Cruz, Calif.) and RPLP1 (polyclonal, Sigma, St. Louis, Mo.) were used. Immunoblots were developed using Super Signal Femto Chemiluminescence (Pierce, Rockford, Ill.) and results were visualized and quantified using the Alpha Innotech (San Leandro, Calif.) imaging system. Monoclonal anti-α tubulin (clone AA13, Santa Cruz Biotechnology, Inc.) was used to detect tubulin expression for normalization of expression of PPIB or RPLP1.

### Example 5

### This Example Illustrates that Cyclophilin B Protein Expression is Associated with Patient Outcome Following Radiation

Given that PPIB RNA expression was strongly correlated to radioresistance (FIG. 7) the inventors evaluated the role of PPIB protein expression as a predictor of clinical outcome following radiation treatment of patients with HNSCC at the University of Virginia (Table 1A). PPIB protein levels were found to predict clinical outcome in these patients (FIG. 5A, B).

Interestingly, expression of CDKN2A (p16), a cyclin-dependent kinase inhibitor and surrogate marker of HPV infection was recently found to predict radiation response in patients with HNCCC. Because this gene was part of the signaling network associated with our 41-gene GEM (Table 3), the inventors sought to determine whether its level of protein expression provided additional predictive ability when combined with that of PPIB. IHC evaluation revealed that p16 levels provided significant stratification of patients with high PPIB IHC (FIGS. 5, C and D) levels supporting relevance of the 39-gene network in radiosensitivity of human cancer.

Those skilled in the art will appreciate, or be able to ascertain using no more than routine experimentation, further features and advantages of the invention based on the above-described embodiments. Accordingly, the invention is not to be limited by what has been particularly shown and described, except as indicated by the appended claims. All publications and references are herein expressly incorporated by reference in their entirety.

TABLE 1A
Patient and Cell Line Data Sets Used in Current Study.

| Dataset Role and | | Total | Radiation | | |
|---|---|---|---|---|---|
| Name | Disease State | (N) | Only (N) | Profiling Type | Reference |
| Training Sets | | | | | |
| BLA-40 | Bladder Cancer Cell | 40 | 39 | Affymetrix HG- | (37) |
| | Lines | | | U133A | |
| Smith | Bladder Tumor Patient | 85 | 0 | Affymetrix HG- | (49, 50) |
| | Samples | | | U133A | |
| Model | | | | | |
| Optimization Set | | | | | |
| Brock | Primary human skin | 16 | 16 | Illumina WG6 | Current |
| | fibroblasts from | | | Expression | Manuscript |
| | radiotherapy patients | | | BeadChip | |
| Test Sets | | | | | |
| NCI-60 | 60 human cancer lines | 60 | 60 | | (16) |
| Rickman | Head and Neck | 81 | 73 | Affymetrix HG- | (51) |
| | Squamous Cell | | | U133 Plus 2.0 | |
| | Carcinoma (HNSCC) | | | | |
| | Patient Samples | | | | |
| Shedden | Lung Adenocarcinoma | 65 | 20 | Affymetrix HG- | (52) |
| | Patient Samples | | | U133A | |
| Virginia | Head and Neck | 72 | 35 | Immunohistochemistry | Current |
| | Squamous Cell | | | for Cyclophillin B | Manuscript |
| | Carcinoma (HNSCC) | | | and p16 | |
| | Patient Samples | | | | |

TABLE 1B
Rickman Head and Neck Squamous Cell Carcinoma Patient
Characteristics (Percentages may not add up to 100 due to rounding)

| | | | No | | | |
|---|---|---|---|---|---|---|
| | | | Chemotherapy | | Chemotherapy | |
| | All (n = 81) | | (n = 73)* | | (n = 8) | |
| Median Age | 59 | (35-79) | 58 | (35-79) | 60 | (43-71) |
| Gender | | | | | | |
| Male | 76 | (94%) | 68 | (93%) | 8 | (100%) |
| Female | 5 | (6%) | 5 | (7%) | 0 | |
| Pathological T Stage | | | | | | |
| T1 | 3 | (4%) | 3 | (4%) | 0 | |
| T2 | 38 | (47%) | 32 | (44%) | 6 | (75%) |
| T3 | 30 | (37%) | 29 | (40%) | 1 | (12.5%) |
| T4 | 10 | (12%) | 9 | (12%) | 1 | (12.5%) |
| Pathological | | | | | | |
| N Stage | | | | | | |
| N0 | 17 | (21%) | 17 | (23%) | 0 | |
| N1 | 15 | (19%) | 15 | (21%) | 0 | |
| N2a | 1 | (1%) | 0 | | 1 | (12.5%) |
| N2b | 26 | (32%) | 24 | (33%) | 2 | (25%) |
| N2c | 14 | (17%) | 12 | (16%) | 2 | (25%) |
| N3 | 8 | (10%) | 5 | (7%) | 3 | (37.5%) |
| Pathological Stage | | | | | | |
| 2 | 7 | (9%) | 7 | (10%) | 0 | |
| 3 | 23 | (28%) | 20 | (27%) | 3 | (37.5%) |
| 4 | 51 | (63%) | 46 | (63%) | 5 | (62.5%) |
| Grade | | | | | | |
| 1 | 19 | (23%) | 18 | (25%) | 1 | (12.5%) |
| 2 | 38 | (47%) | 33 | (45%) | 5 | (62.5%) |
| 3 | 24 | (30%) | 22 | (30%) | 2 | (25%) |
| HPV Status | | | | | | |
| HPV free | 75 | (93%) | 67 | (92%) | 8 | (100%) |
| Undetermined | 6 | (7%) | 6 | (8%) | 0 | |
| Localization | | | | | | |
| Lip | 18 | (22%) | 16 | (22%) | 2 | (25%) |
| Mouth | 10 | (12%) | 10 | (14%) | 0 | |
| Oropharynx | 17 | (21%) | 16 | (22%) | 1 | (12.5%) |
| Pharynx | 36 | (44%) | 31 | (42%) | 5 | (62.5%) |
| Overall Survival | | | | | | |
| Alive | 29 | (36%) | 28 | (38%) | 1 | (12.5%) |
| Dead | 50 | (62%) | 44 | (60%) | 6 | (75%) |
| Unknown | 2 | (2%) | 1 | (1%) | 1 | (12.5%) |
| Median (mos) | 55 | (7-158) | 59 | (7-158) | 36 | (16-151) |
| Metastasis Free | | | | | | |
| Survival | | | | | | |
| Metastatic | 40 | (49%) | 34 | (47%) | 7 | (87.5%) |
| Nonmetastatic | 41 | (51%) | 39 | (53%) | 1 | (12.5%) |
| Median (mos) | 37 | (3-158) | 43 | (3-158) | 19 | (5-151) |

*Only 72 of the 73 had complete data.

TABLE 1C
Shedden Lung Adenocarcinoma Patient Characteristics (24)
(Percentages may not add up to 100 due to rounding)

| | | | No | | | |
|---|---|---|---|---|---|---|
| | | | chemotherapy | | Chemotherapy | |
| | All (n = 65) | | (n = 20) | | (n = 45) | |
| Median Age | 64 | (36-82) | 68 | (58-82) | 62 | (36-82) |
| Gender | | | | | | |
| Male | 22 | (34%) | 6 | (30%) | 16 | (35%) |
| Female | 43 | (66%) | 14 | (70%) | 29 | (64%) |
| Race | | | | | | |
| White | 56 | (86%) | 17 | (85%) | 39 | (87%) |
| Black | 3 | (5%) | 3 | (15%) | 0 | |
| Asian | 1 | (2%) | 0 | | 1 | (2%) |
| Unknown | 5 | (8%) | 0 | | 5 | (11%) |
| Smoking History | | | | | | |
| Current Smoker | 3 | (5%) | 0 | | 3 | (7%) |
| Former Smoker | 50 | (77%) | 19 | (95%) | 31 | (69%) |
| Never Smoker | 10 | (15%) | 1 | (5%) | 9 | (20%) |
| Unknown | 2 | (3%) | 0 | | 2 | (4%) |
| Pathological T Stage | | | | | | |
| T1 | 12 | (18%) | 3 | (15%) | 9 | (20%) |
| T2 | 43 | (66%) | 10 | (50%) | 33 | (73%) |
| T3 | 8 | (12%) | 6 | (30%) | 2 | (4%) |
| T4 | 1 | (2%) | 1 | (5%) | 0 | |
| NA | 1 | (2%) | 0 | | 1 | (2%) |
| Pathological | | | | | | |
| N Stage | | | | | | |
| NO | 27 | (42%) | 11 | (55%) | 16 | (36%) |
| N1 | 15 | (23%) | 5 | (25%) | 10 | (22%) |
| N2 | 22 | (34%) | 4 | (20%) | 18 | (40%) |
| NA | 1 | (2%) | 0 | | 1 | (2%) |
| Histologic Grade | | | | | | |
| Well Differentiated | 4 | (6%) | 2 | (10%) | 2 | (4%) |
| Moderately | 30 | (46%) | 5 | (5%) | 25 | (56%) |
| Differentiated | | | | | | |
| Poorly | 29 | (45%) | 13 | (65%) | 16 | (36%) |
| Differentiated | | | | | | |
| NA | 2 | (3%) | 0 | | 2 | (4%) |
| Surgical Margins | | | | | | |
| Negative | 61 | (94%) | 17 | (85%) | 44 | (98%) |
| Positive | 3 | (5%) | 3 | (15%) | 0 | |
| NA | 1 | (2%) | 0 | | 1 | (2%) |
| Overall Survival | | | | | | |
| Alive | 14 | (22%) | 3 | (15%) | 11 | (24%) |
| Dead | 51 | (78%) | 17 | (85%) | 34 | (76%) |
| Median (mos) | 40 | (2-132.5) | 28.25 | (2-88) | 42 | (8.7-132.5) |
| Relapse-Free | | | | | | |
| Survival | | | | | | |
| Relapsed | 53 | (82%) | 14 | (70%) | 39 | (87%) |
| No relapse | 9 | (14%) | 3 | (15%) | 6 | (13%) |
| Unknown | 3 | (5%) | 3 | (15%) | 0 | |
| Median | 13 | (1-94) | 13.62 | (1-55) | 13 | (1-94) |

TABLE 2A
Survival Fraction Values after 2 Gy ionizing radiation (SF2)
of human bladder cancer (BLA-40, Table 1A).

| | Mean | |
|---|---|---|
| Bladder Cell | Survival | Standard |
| Line | Fraction | Deviation |
| 253J-BV | 0.461 | 0.061 |
| 253JLaval | 0.506 | 0.035 |
| 253J-P | 0.462 | 0.083 |
| 575A | 0.481 | 0.017 |
| BC16.1 | 0.500 | 0.046 |
| CRL2169 | 0.948 | 0.065 |
| CRL2742 | 0.657 | 0.033 |
| CRL7193 | 0.506 | 0.106 |
| CUBIII | 0.721 | 0.041 |
| FL3 | 0.974 | 0.023 |
| HT1197 | 0.717 | 0.065 |
| HT1376 | 0.239 | 0.032 |
| HTB9 | 0.603 | 0.022 |
| HU456 | 0.376 | 0.004 |
| J82 | 0.524 | 0.041 |
| JON | 0.297 | 0.047 |
| KK47 | 0.768 | 0.072 |
| KU7 | 0.515 | 0.036 |
| MGH-U3 | 0.486 | 0.049 |
| MGH-U4 | 0.597 | 0.067 |
| PSI | 0.494 | 0.082 |
| RT4 | 0.800 | 0.035 |
| SCaBER | 0.603 | 0.100 |
| SLT4 | 0.468 | 0.064 |
| SW1710 | 0.588 | 0.076 |
| T24 | 0.626 | 0.089 |
| T24T | 0.319 | 0.055 |
| TCCSUP | 0.198 | 0.021 |
| UMUC1 | 0.846 | 0.069 |
| UMUC13D | 0.973 | 0.026 |
| UMUC14 | 0.778 | 0.029 |
| UMUC2 | 0.523 | 0.052 |
| UMUC3 | 0.969 | 0.031 |
| UMUC3-E | 0.460 | 0.025 |
| UMUC6 | 0.360 | 0.041 |
| UMUC9 | 0.633 | 0.110 |
| VMCUB1 | 0.398 | 0.036 |
| VMCUB2 | 0.870 | 0.085 |
| VMCUB3 | 0.509 | 0.063 |

TABLE 2B
Survival Fraction Values after absorbing 2 Gy ionizing radiation
of primary human skin fibroblast (HSF) cell lines developed from
skin biopsies (Brock, Table 1A)

| HNSCC Cell | |
|---|---|
| Line | Survival Fraction |
| C28 | 0.300 |
| C29 | 0.330 |
| C34 | 0.288 |
| C38 | 0.339 |
| C39 | 0.361 |
| C42 | 0.167 |
| C43 | 0.423 |
| C56 | 0.194 |
| C66 | 0.333 |
| C68 | 0.302 |
| C69 | 0.303 |
| C74 | 0.175 |
| C80 | 0.410 |
| S10 | 0.437 |
| S34 | 0.140 |
| S38 | 0.131 |

TABLE 3
The 41 genes corresponding to probe sets in the optimal GEM of cellular
response to radiation

| Gene | | | Molecular | |
|---|---|---|---|---|
| Symbol | Probe ID | Gene Name | Function* | Biological Process* |
| ACADVL | 200710_at | acyl-Coenzyme A | Dehydrogenase | Acyl-CoA metabolism; |
| | ILMN_1806408 | dehydrogenase, very | | Electron transport |
| | | long chain | | |
| AP3M2 | 203410_at | adaptor-related protein | Other membrane | Pinocytosis; Transport |
| | ILMN_1676946 | complex 3, mu 2 | traffic protein | |
| | | subunit | | |
| ATP5F1 | 211755_s_at | ATP synthase, H+ | Hydrogen | Cation transport |
| | ILMN_1721989 | transporting, | transporter; Synthase; | |
| | | mitochondrial F0 | Other hydrolase | |
| | | complex, subunit B1 | | |
| BLK | 206255_at | B lymphoid tyrosine | Non-receptor tyrosine | Carbohydrate transport; |
| | ILMN_1668277 | kinase | protein kinase | Protein phosphorylation; |
| | | | | Intracellular signaling |
| | | | | cascade; Transport; |
| | | | | Immunity and defense; |
| | | | | Embryogenesis; |
| | | | | Neurogenesis; Mesoderm |
| | | | | development; Cell cycle |
| | | | | control; Cell proliferation |
| | | | | and differentiation; |
| | | | | Oncogene |
| C17orf62 | 218130_at | chromosome 17 open | Molecular function | Biological process |
| | ILMN_1750401 | reading frame 62 | unclassified | unclassified |
| C19orf66 | 53720_at | hypothetical protein | Molecular function | Biological process |
| | ILMN_1750400 | FLJ11286 | unclassified | unclassified |
| CCDC76 | 219130_at | coiled-coil domain | Molecular function | Biological process |
| | ILMN_1659786 | containing 76 | unclassified | unclassified |
| CFLAR | 211317_s_at | CASP8 and FADD- | Cysteine protease | Proteolysis; Apoptosis |
| | ILMN_1789830 | like apoptosis | | |
| | | regulator | | |
| CLNS1A | 209143_s_at | chloride channel, | Other transporter | Anion transport |
| | ILMN_1736814 | nucleotide-sensitive, | | |
| | | 1A | | |
| CLPX | 204809_at | ClpX caseinolytic | Other chaperones | Protein folding; |
| | ILMN_1709894 | peptidase X homolog | | Proteolysis; Transport |
| | | (E. coli) | | |
| CREB3 | 209432_s_at | cAMP responsive | CREB transcription | mRNA transcription |
| | ILMN_1703072 | element binding | factor; Nucleic acid | regulation |
| | | protein 3 | binding | |
| DNM3 | 209839_at | dynamin 3 | Microtubule family | Endocytosis; Transport; |
| | ILMN_1680928 | | cytoskeletal protein; | Cell structure |
| | | | Small GTPase; Other | |
| | | | hydrolase | |
| GPRC5A | 203108_at | G protein-coupled | Molecular function | Biological process |
| | ILMN_1682599 | receptor, family C, | unclassified | unclassified |
| | | group 5, member A | | |
| IL15 | 205992_s_at | interleukin 15 | Interleukin | Cytokine and chemokine |
| | ILMN_1724181 | | | mediated signaling |
| | | | | pathway; MAPKKK |
| | | | | cascade; JAK-STAT |
| | | | | cascade; Ligand-mediated |
| | | | | signaling; Immunity and |
| | | | | defense; Inhibition of |
| | | | | apoptosis |
| INVS | 210114_at | inversin | Molecular function | Proteolysis; Cell surface |
| | ILMN_1763137 | | unclassified | receptor mediated signal |
| | | | | transduction |
| IRAK4 | 219618_at | interleukin-1 receptor- | Serine/threonine | Protein phosphorylation; |
| | ILMN_1692352 | associated kinase 4 | protein kinase | Receptor protein |
| | | | receptor; Non- | serine/threonine kinase |
| | | | receptor | signaling pathway; |
| | | | serine/threonine | Immunity and defense |
| | | | protein kinase | |
| LBA1 | 213261_at | lupus brain antigen 1 | Molecular function | Biological process |
| | ILMN_1750321 | | unclassified | unclassified |
| MIS12 | 221559_s_at | MIS12, MIND | Molecular function | Biological process |
| | ILMN_1718069 | kinetochore complex | unclassified | unclassified |
| | | component, homolog | | |
| | | (yeast) | | |
| MRPL13 | 218049_s_at | mitochondrial | Ribosomal protein | Protein biosynthesis |
| | ILMN_1671158 | ribosomal protein L13 | | |
| NFKBIE | 203927_at | nuclear factor of kappa | Molecular function | Biological process |
| | ILMN_1717313 | light polypeptide gene | unclassified | unclassified |
| | | enhancer in B-cells | | |
| | | inhibitor, epsilon | | |
| NOS3 | 205581_s_at | nitric oxide synthase 3 | Synthase; | Electron transport; Nitric |
| | ILMN_1775224 | (endothelial cell) | Oxidoreductase; | oxide biosynthesis; NO |
| | | | Calmodulin related | mediated signal |
| | | | protein | transduction; Other |
| | | | | metabolism |
| OLA1 | 219293_s_at | GTP-binding protein 9 | G-protein | Biological process |
| | ILMN_1659820 | (putative) | | unclassified |
| PALM | 203859_s_at | paralemmin | Other miscellaneous | Signal transduction |
| | ILMN_1812031 | | function protein | |
| PBLD | 219543_at | phenazine | Oxidoreductase | Other metabolism |
| | ILMN_1713319 | biosynthesis-like | | |
| | | protein domain | | |
| | | containing | | |
| PPIB | 200967_at | peptidylprolyl | Other isomerase | Protein folding; Nuclear |
| | ILMN_1711745 | isomerase B | | transport; Immunity and |
| | | (cyclophilin B) | | defense |
| PRRG1 | 205618_at | proline rich Gla (G- | Molecular function | Biological process |
| | ILMN_1781791 | carboxyglutamic acid) 1 | unclassified | unclassified |
| PSMB9 | 204279_at | proteasome (prosome, | Other proteases | Proteolysis |
| | ILMN_1798233 | macropain) subunit, | | |
| | | beta type, 9 (large | | |
| | | multifunctional | | |
| | | peptidase 2) | | |
| PSMG2 | 218467_at | tumor necrosis factor | Other nucleic acid | Other apoptosis; Induction |
| | ILMN_1797445 | superfamily, member | binding | of apoptosis; Other |
| | | 5-induced protein 1 | | developmental process |
| RNF115 | 212742_at | zinc finger protein 364 | Molecular function | Biological process |
| | ILMN_1811997 | | unclassified | unclassified |
| RPL8 | 200936_at | ribosomal protein L8 | Other RNA-binding | Protein biosynthesis |
| | ILMN_1764721 | | protein; Ribosomal | |
| | | | protein | |
| RPLP1 | 200763_s_at | ribosomal protein, | Ribosomal protein | Protein biosynthesis |
| | ILMN_1689725 | large, P1 | | |
| SEPT7 | 213151_s_at | septin 7 | Cytoskeletal protein; | Cytokinesis |
| | ILMN_1729019 | | Small GTPase | |
| SETD3 | 212465_at | SET domain | Molecular function | Biological process |
| | ILMN_1724504 | containing 3 | unclassified | unclassified |
| STAT4 | 206118_at | signal transducer and | Other transcription | Biological process |
| | ILMN_1785202 | activator of | factor; Nucleic acid | unclassified |
| | | transcription 4 | binding | |
| TGDS | 208249_s_at | TDP-glucose 4,6- | Dehydratase; | Glycogen metabolism |
| | ILMN_1685567 | dehydratase | Epimerase/racemase | |
| TMEM135 | 222209_s_at | transmembrane | Molecular function | Biological process |
| | ILMN_1700202 | protein 135 | unclassified | unclassified |
| TMEM70 | 219448_at | transmembrane | Molecular function | Biological process |
| | ILMN_1739032 | protein 70 | unclassified | unclassified |
| TNFAIP1 | 201207_at | tumor necrosis factor, | Molecular function | Biological process |
| | ILMN_1655429 | alpha-induced protein | unclassified | unclassified |
| | | 1 (endothelial) | | |
| TNS3 | 217853_at | tensin 3 | Protein phosphatase; | Phospholipid metabolism; |
| | ILMN_1667893 | | Other phosphatase | Protein phosphorylation; |
| | | | | Cell adhesion; Immunity |
| | | | | and defense; Induction of |
| | | | | apoptosis; Cell cycle |
| | | | | control; Cell |
| | | | | differentiation; Tumor |
| | | | | suppressor |
| WDYHV1 | 219060_at | chromosome 8 open | Molecular function | Biological process |
| | ILMN_1695491 | reading frame 32 | unclassified | unclassified |
| ZNF7 | 205089_at | zinc finger protein 7 | KRAB box | mRNA transcription |
| | ILMN_1784281 | | transcription factor | regulation; Cell |
| | | | | proliferation and |
| | | | | differentiation |

PANTHER Classification System database at pantherdb.org

TABLE 4
Cox Proportional Hazards Regression Model Analysis.
A: Cox proportional hazards regression model analysis for overall
survival in the Rickman HNSCC patient dataset, (N = 72).
AIC-based model selection.

| | coef | exp(coef) | se(coef) | z | p |
|---|---|---|---|---|---|
| T stage | 0.310 | 0.733 | 0.218 | 1.42 | 0.160 |
| N stage | 0.700 | 2.013 | 0.422 | 1.66 | 0.097 |
| COXEN GEM score* | −0.989 | 0.372 | 0.497 | −1.99 | 0.047 |

Likelihood ratio test = 9.23 on 3 df, p = 0.0264 n = 72
*COXEN GEM score is directly related to radiosensitivity

B: Cox proportional hazards regression model analysis for distant
metastasis-free survival time in the Rickman HNSCC patient dataset
(N = 72). AIC-based model selection.

| | coef | exp(coef) | se(coef) | Z | P |
|---|---|---|---|---|---|
| COXEN GEM score* | −1.03 | 0.356 | 0.565 | −1.83 | 0.068 |

Likelihood ratio test = 3.34 on 1 df, p = 0.0676 n = 72
*COXEN GEM score is directly related to radiosensitivity

C: Cox proportional hazards regression model analysis for overall
survival in the SheddenLung Adenocarcinoma patient dataset, for all
patients (N = 63). AIC-based model selection.

| | coef | exp(coef) | se(coef) | z | P |
|---|---|---|---|---|---|
| Chemotherapy | −1.11 | 0.329 | 0.334 | −3.33 | 0.00086 |
| N stage | 0.68 | 1.974 | 0.178 | 3.81 | 0.00014 |
| COXEN GEM score* | −1.17 | 0.310 | 0.584 | −2.01 | 0.04500 |

Likelihood ratio test = 20.1 on 3 df, p = 0.000158 n = 63
*COXEN GEM score is directly related to radiosensitivity

D: Cox proportional hazards regression model analysis for overall
survival in the Shedden Lung Adenocarcinoma dataset, for the twenty
patients who only received radiotherapy (N = 20). AIC-based
model selection.

| | coef | exp(coef) | se(coef) | z | P |
|---|---|---|---|---|---|
| N stage | 0.594 | 1.810 | 0.293 | 2.03 | 0.043 |
| COXEN GEM score* | −1.827 | 0.161 | 0.962 | −1.90 | 0.057 |

Likelihood ratio test = 7.39 on 2 df, p = 0.0248 n = 20
*COXEN GEM score is directly related to radiosensitivity

What is claimed is:

1-14. (canceled)

15. A method of assessing the efficacy or effectiveness of a radiation treatment being administered to a cancer subject, the method comprising comparing:
a) the expression level of a marker measured in a first sample obtained from the subject at a time t₀, wherein the marker is selected from the group consisting of:
i) a marker gene having at least 95% sequence identity with Cyclophilin B (PPIB) gene, or homologs or variants thereof;
ii) a marker gene having at least 95% sequence identity with Acidic Ribosomal Phosphoprotein P1 (RPLP1) gene, or homologs or variants thereof
iii) a plurality of marker genes comprising a marker gene having at least 95% sequence identity to PPIB gene and another marker gene having at least 95% sequence identity to CDKN2A gene, or homologs or variants thereof;
iv) a plurality of marker genes comprising a marker gene having at least 95% sequence identity with PPIB gene or RRLP1 gene or both, and at least one marker gene having at least 95% sequence identity with a sequence selected from table 3, or homologs or variants thereof;
v) a plurality of marker genes comprising a marker gene having at least 95% sequence identity with PPIB, a marker gene having at least 95% sequence identity with CDKN2A and at least one marker gene having at least 95% sequence identity with a sequence selected from Table 3, or homologs or variants thereof;
vi) a plurality of marker genes having at least 95% sequence identity with a sequence selected from table 3, or homologs or variants thereof;
vii) a polynucleotide which is fully complementary to at least a portion of a marker gene of i)-vi);
viii) polypeptides encoded by the marker genes of i)-vi); and
ix) fragments of polypeptides of viii);
b) the level of the marker in a second sample obtained from the subject at time t₁; and,
wherein a change in the level of the marker in the second sample relative to the first sample is an indication that the radiation treatment is effective for treating cancer in the subject.

16. The method of claim 15, wherein the plurality of marker genes comprises a gene having at least 95% sequence identity with PPIB gene, or homologs or variants thereof; and wherein a decrease in the expression level of the PPIB is an indication that the radiation treatment is effective for treating cancer in the subject.

17. The method of claim 16, wherein the plurality of marker genes further comprises a gene having at least 95% sequence identity to CDKN2A gene, or homologs or variants thereof; and wherein an increase in the expression level of CDKN2A gene is an indication that the radiation treatment is effective for treating cancer in the subject.

18. The method of claim 15, wherein the plurality of marker genes comprises a gene having at least 95% sequence identity with RPLP1 gene, or homologs or variants thereof; and wherein a decrease in the expression level of the RPLP1 is an indication that the radiation treatment is effective for treating cancer in the subject.

19. The method of claim 15, wherein the genes detected share 100% sequence identity with the corresponding marker genes in i)-vi).

20. The method of claim 15, wherein the time t₀ is before the treatment has been administered to the subject, and the time t₁ is after the treatment has been administered to the subject.

21. The method of claim 15, wherein the comparing is repeated over a range of times.

22-24. (canceled)

25. The method of claim 15, wherein the presence of the marker is determined by obtaining RNA from the cancer tissue sample; generating cDNA from the RNA; amplifying the cDNA with probes or primers for marker genes; obtaining from the amplified cDNA the expression levels of the genes or gene expression products in the sample.

26. (canceled)

27. An assay system for predicting patient response or outcome to radiation therapy for cancer comprising a means to detect the expression of a marker gene or plurality of marker genes selected from the group consisting of:
i) a marker gene having at least 95% sequence identity with Cyclophilin B (PPIB) gene, or homologs or variants thereof;
ii) a marker gene having at least 95% sequence identity with Acidic Ribosomal Phosphoprotein P1 (RPLP1) gene, or homologs or variants thereof
iii) a plurality of marker genes comprising a marker gene having at least 95% sequence identity to PPIB gene and another marker gene having at least 95% sequence identity to CDKN2A gene, or homologs or variants thereof;
iv) a plurality of marker genes comprising a marker gene having at least 95% sequence identity with PPIB gene or RRLP1 gene or both, and at least one marker gene having at least 95% sequence identity with a sequence selected from table 3, or homologs or variants thereof;
v) a plurality of marker genes comprising a marker gene having at least 95% sequence identity with PPIB, a marker gene having at least 95% sequence identity with CDKN2A and at least one marker gene having at least 95% sequence identity with a sequence selected from Table 3, or homologs or variants thereof;
vi) a plurality of marker genes having at least 95% sequence identity with a sequence selected from table 3, or homologs or variants thereof;
vii) a polynucleotide which is fully complementary to at least a portion of a marker gene of i)-vi).

28. The assay system of claim 27, wherein the genes detected share 100% sequence identity with the corresponding marker gene in i)-vi).

29. (canceled)

30. The assay system of claim 27, wherein the means to detect comprises binding ligands that specifically detect polypeptides encoded by the marker genes.

31. The assay system of claim 27, wherein the means to detect comprises at least one of nucleic acid probes and binding ligands disposed on an assay surface.

32. The assay system of claim 31, wherein the assay surface comprises a chip, array, or fluidity card.

33. (canceled)

34. (canceled)

35. The assay system of claim 27, further comprising: a control selected from the group consisting of:
information containing a predetermined control level of the marker gene that has been correlated with response to the administration of radiation therapy; and
information containing a predetermined control level of the marker gene that has been correlated with a lack of response to the administration of radiation therapy.

36. (canceled)

37. The method of claim 36, wherein the agent is selected from the group consisting of: a PPIB synthetic inhibitor, a nucleic acid molecule, an antibody or a biologically active fragment thereof, and an aptamer.

38. The method of claim 37, wherein the nucleic acid molecule is selected from the group consisting of an anti-sense oligonucleotide, an RNAi construct, a DNA enzyme, and a ribozyme that specifically inhibits the expression of PPIB.

39. The method of claim 37, wherein the antibody or a biologically active fragment thereof specifically binds to PPIB.

40. (canceled)

41. The method of claim 36, wherein the radiation therapy is combined with an anti-cancer therapy selected from the group consisting of surgery and chemotherapy.

42-44. (canceled)
